(19) United States
(12) Patent Application Publication
(10) Pub. No.: US 2008/0021045 A1
(43) Pub. Date: Jan. 24, 2008

(54) 7-Amino-5-Halopyrazolopyrimidines with a Fungicidal Action

(75) Inventors: Olaf Gebauer, Leverkusen (DE); Ulrich Heinemann, Leichlingen (DE); Stefan Herrmann, Leichlingen (DE); Herbert Gayer, Monheim (DE); Jorg Nico Greul, Leichlingen (DE); Stefan Hillebrand, Neuss (DE); Hans-Ludwig Elbe, Wuppertal (DE); Ronald Ebbert, Nurnberg (DE); Ulrike Wachendorff-Neumann, Neuwied (DE); Peter Dahmen, Neuss (DE); Karl-Heinz Kuck, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: BAYER CROPSCIENCE AKTIENGESELLSCHSFT, Monheim (DE)

(21) Appl. No.: 10/581,891

(22) PCT Filed: Dec. 8, 2004
(86) PCT No.: PCT/EP04/13939

§ 371 Date: Apr. 11, 2007

(30) Foreign Application Priority Data
Dec. 10, 2003 (DE) 103 57 569.3

(51) Int. Cl. C07D 487/04; A01N 43/54; A01P 1/00; A01P 3/00

(52) U.S. Cl. 514/259.1; 544/281

(57) ABSTRACT

Novel pyrazolopyrimidines of the formula

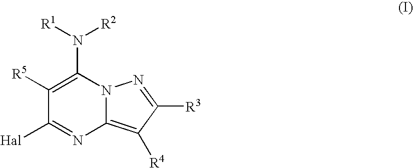

in which
R¹, R², R³, R⁴, R⁵ and Hal are as defined in the description,
a plurality of processes for preparing these compounds and their use for controlling unwanted microorganisms.

The invention relates to pyrazolopyrimidines, to a plurality of processes for their preparation and to their use for controlling unwanted microorganisms.

It is already known that certain pyrazolopyrimidines have fungicidal properties (compare DE-A 3 130 633 or FR-A 2 794 745).

However, since the ecological and economical demands made on modem fungicides are increasing constantly, for example with respect to activity spectrum, toxicity, selectivity, application rate, formation of residues and favorable manufacture, and there can furthermore be problems, for example, with resistance, there is a constant need to develop novel fungicides which, at least in some areas, have advantages over those of the prior art.

This invention now provides novel pyrazolopyrimidines of the formula

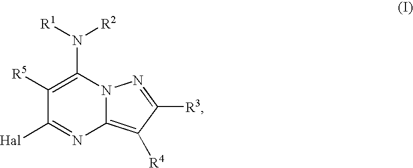

in which

R¹ represents optionally substituted alkyl, optionally substituted alkenyl, optionally substituted alkinyl, optionally substituted cycloalkyl or represents optionally substituted heterocyclyl,

R² represents hydrogen or alkyl, or

R¹ and R² together with the nitrogen atom to which they are attached represent an optionally substituted heterocyclic ring,

R³ represents hydrogen, halogen, optionally substituted alkyl or optionally substituted cycloalkyl,

R⁴ represents halogen, cyano, nitro, alkyl, hydroxyalkyl, alkoxyalkyl, haloalkyl, cycloalkyl, formyl, thiocarbamoyl, alkoxycarbonyl, alkylcarbonyl, benzylcarbonyl, cycloalkylcarbonyl, hydroxyiminoalkyl, alkoximinoalkyl, alkylthio, alkylsulfinyl, alkylsulfonyl or alkylaminocarbonyl,

Hal represents halogen and

R⁵ represents alkyl, haloalkyl, alkenyl, haloalkenyl, cycloalkyl, halogen- or alkyl-substituted cycloalkyl, cycloalkenyl or represents halogen- or alkyl-substituted cycloalkenyl.

Furthermore, it has been found that pyrazolopyrimidines of the formula (1) can be prepared when
(a) halopyrazolopyrimidines of the formula

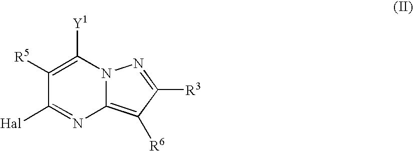

in which
R³, R⁵ and Hal are as defined above,
R⁶ represents halogen, cyano, nitro, alkyl, haloalkyl, cycloalkyl, formyl, thio-carbamoyl, alkoxycarbonyl, alkylthio, alkylsulfinyl, alkylsulfonyl or alkylaminocarbonyl and
Y¹ represents halogen,
are reacted with amines of the formula

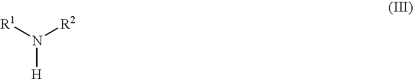

in which
R¹ and R² are as defined above,
if appropriate in the presence of a diluent, if appropriate in the presence of a catalyst and if appropriate in the presence of an acidic receptor, or
b) pyrazolopyrimidines of the formula

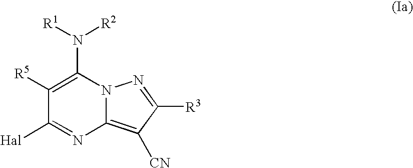

in which
R¹, R², R³, R⁵ and Hal are as defined above
are either
α) reacted with diisobutylaluminum hydride in the presence of aqueous ammonium chloride solution and in the presence of an organic diluent, or
β) reacted with Grignard compounds of the formula

R⁷—Mg—X² (IV)
in which
R⁷ represents alkyl, benzyl or cycloalkyl and
X² represents chlorine, bromine or iodine,
in the presence of a diluent and, if appropriate, in the presence of a catalyst, or
c) pyrazolopyrimidines of the formula

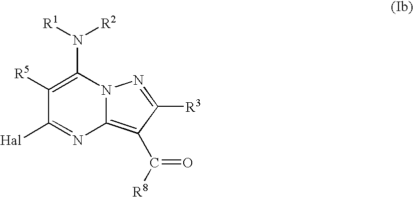

in which
R¹, R², R³, R⁵ and Hal are as defmed above and
R⁸ represents hydrogen, alkyl, benzyl or cycloalkyl,
are either
α) reacted with amino compounds of the formula

H₂N—OR⁹ (V)
in which
R⁹ represents hydrogen or alkyl,
in the presence of a diluent and, if appropriate, in the presence of a catalyst, where the amino compounds of the formula (V) can also be employed in the form of their acid addition salts, or
β) reacted with diisobutylaluminum hydride in the presence of aqueous ammonium chloride solution and in the presence of an organic diluent, or reacted with sodium borohydride in the presence of a diluent,
and the resulting pyrazolopyrimidines of the formula

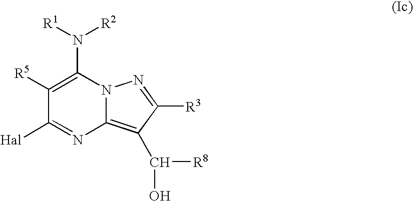

in which
R¹, R², R³, R⁵ R⁸ and Hal are as defined above are, if appropriate, reacted with alkylating agents of the formula

R¹⁰—X¹ (VI)
in which
R¹⁰ represents alkyl and
X¹ represents chlorine, bromine, iodine or the radical R¹⁰O—SO₂—O—,
if appropriate in the presence of a base and in the presence of a diluent, or
d) pyrazolopyrimidines of the formula

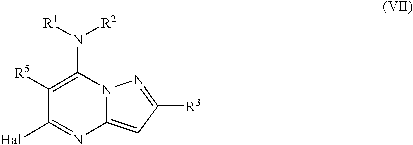

in which
R¹, R², R³, R⁵ and Hal are as defined above
are reacted with acid halides of the formula

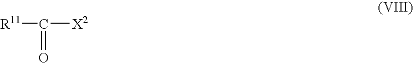

in which
R¹¹ represents alkyl, benzyl or cycloalkyl and
X² represents chlorine or bromine, or
with acid anhydrides of the formula

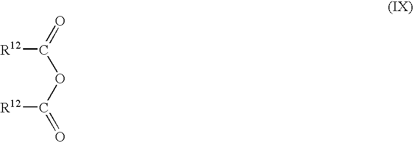

in which
R¹² represents alkyl,
   in each case in the presence of a catalyst and in the presence of a diluent.

Finally, it has been found that the pyrazolopyrimidines of the formula (I) are highly suitable for controlling unwanted microorganisms. Especially, they have strong fungicidal activity and can be used both in crop protection and in the protection of materials.

Surprisingly, the pyrazolopyrimidines of the formula (I) according to the invention have a considerably better microbicidal activity than the constitutionally most similar prior-art compounds of the same direction of action.

Depending on the substitution pattern, the compounds according to the invention can, if appropriate, be present as mixtures of different possible isomeric forms, in particular of stereoisomers, such as E and Z, threo and erythro and also optical isomers, and, if appropriate, also in the form of tautomers. If R⁵ has different substituents on the two atoms adjacent to the point of attachment, the compounds in question can be present in a particular stereoisomeric form, i.e. atropisomers.

The formula (I) provides a general definition of the pyrazolopyrimidines according to the invention. Preference is given to those compounds of the formula (I) in which
R¹ represents alkyl having 1 to 6 carbon atoms which may be mono- to pentaisubstituted by identical or different substituents from the group consisting of halogen, cyano, hydroxy, alkoxy having 1 to 4 carbon atoms and cycloalkyl having 3 to 6 carbon atoms, or

R¹ represents alkenyl having 2 to 6 carbon atoms which may be mono- to trisubstituted by identical or different substituents from the group consisting of halogen, cyano, hydroxy, alkoxy having 1 to 4 carbon atoms and cycloalkyl having 3 to 6 carbon atoms, or

R¹ represents alkinyl having 3 to 6 carbon atoms which may be mono- to trisubstituted by identical or different substituents from the group consisting of halogen, cyano, alkoxy having 1 to 4 carbon atoms and cycloalkyl having 3 to 6 carbon atoms, or

R¹ represents cycloalkyl having 3 to 6 carbon atoms which may be mono- to trisubstituted by identical or different substituents from the group consisting of halogen and alkyl having 1 to 4 carbon atoms, or

R¹ represents saturated or unsaturated heterocyclyl having 5 or 6 ring members and 1 to 3 heteroatoms, such as nitrogen, oxygen and/or sulfur, where the heterocyclyl may be mono- or disubstituted by halogen, alkyl having 1 to 4 carbon atoms, cyano, nitro and/or cycloalkyl having 3 to 6 carbon atoms,

R² represents hydrogen or alkyl having 1 to 4 carbon atoms, or

R¹ and R² together with the nitrogen atom to which they are attached represent a saturated or unsaturated heterocyclic ring having 3 to 6 ring members, where the heterocycle may contain a further nitrogen, oxygen or sulfur atom as ring member and where the heterocycle may be substituted up to 3 times by fluorine, chlorine, bromine, alkyl having 1 to 4 carbon atoms and/or haloalkyl having 1 to 4 carbon atoms and 1 to 9 fluorine and/or chlorine atoms,

R³ represents hydrogen, fluorine, chlorine, bromine, iodine, alkyl having 1 to 4 carbon atoms, haloalkyl having 1 to 4 carbon atoms and 1 to 4 halogen atoms or represents cycloalkyl having 3 to 6 carbon atoms,

R⁴ represents cyano, fluorine, chlorine, bromine, iodine, nitro, formyl, haloalkyl having 1 to 4 carbon atoms and 1 to 9 fluorine, chlorine and/or bromine atoms, alkyl having 1 to 4 carbon atoms, hydroxyalkyl having 1 to 4 carbon atoms, alkoxyalkyl having 1 to 4 carbon atoms in the alkoxy moiety and 1 to 4 carbon atoms in the alkyl moiety, cycloalkyl having 3 to 6 carbon atoms, thiocarbomoyl, alkoxycarbonyl having 1 to 4 carbon atoms in the alkoxy moiety, alkylcarbonyl having 1 to 4 carbon atoms in the alkyl moiety, benzylcarbonyl, cycloalkylcarbonyl having 3 to 6 carbon atoms in the cycloalkyl moiety, hydroximinoalkyl having 1 to 4 carbon atoms in the alkyl moiety, alkoximinoalkyl having 1 to 4 carbon atoms in the alkoxy moiety and 1 to 4 carbon atoms in the alkyl moiety, alkylthio having 1 to 4 carbon atoms, alkylsulfmyl having 1 to 4 carbon atoms, alkylsulfonyl having 1 to 4 carbon atoms or represents alkylaminocarbonyl having 1 to 4 carbon atoms in the alkyl moiety,

Hal represents fluorine, chlorine or bromine and

R⁵ represents alkyl having 1 to 6 carbon atoms, alkenyl having 2 to 6 carbon atoms, cycloalkyl having 3 to 8 carbon atoms, cycloalkenyl having 3 to 8 carbon atoms, haloalkyl having 1 to 6 carbon atoms and 1 to 5 fluorine, chlorine and/or bromine atoms, haloalkenyl having 2 to 6 carbon atoms and 1 to 5 fluorine, chlorine and/or bromine atoms, cycloalkyl which has 3 to 8 carbon atoms and is substituted by 1 to 3 fluorine, chlorine and/or bromine atoms or represents cycloalkenyl which has 3 to 8 carbon atoms and is substituted by 1 to 3 fluorine, chlorine and/or bromine atoms or methyl groups.

Particular preference is given to those pyrazolopyrimidines of the formula (I) in which

R¹ represents a radical of the formula

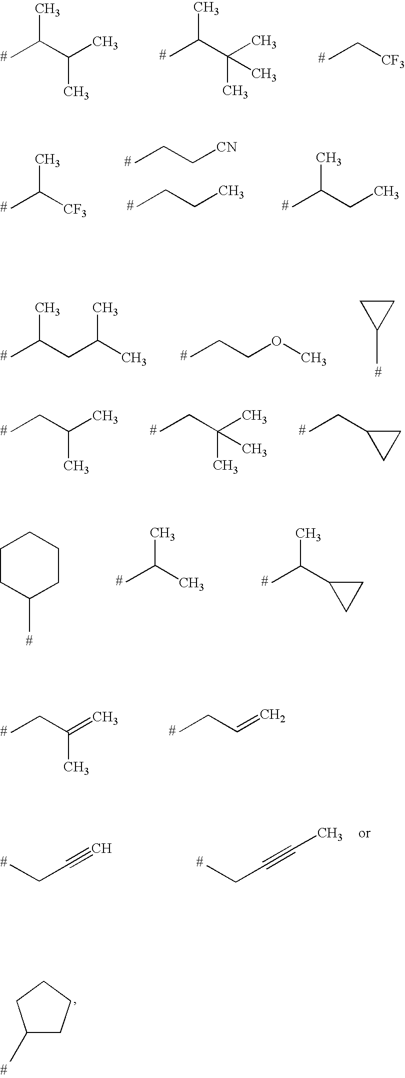

where # denotes the point of attachment,

R² represents hydrogen, methyl, ethyl or propyl, or

R¹ and R² together with the nitrogen atom to which they are attached represent pyrrolidinyl, piperidinyl, morpholinyl, thiomorpholinyl, piperazinyl, 3,6-dihydro-1(2H)-piperidinyl or tetrahydro-1(2H)-pyridazinyl, where these radicals may be substituted by 1 to 3 fluorine atoms, 1 to 3 methyl groups and/or trifluoromethyl, or

R¹ and R² together with the nitrogen atom to which they are attached represent a radical of the formula

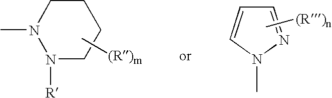

in which

R′ represents hydrogen or methyl,

R″ represents methyl, ethyl, fluorine, chlorine or trifluoromethyl,

m represents the numbers 0, 1, 2 or 3, where R″ represents identical or different radicals, if m represents 2 or 3,

R′″ represents methyl, ethyl, fluorine, chlorine or trifluoromethyl and

n represents the numbers 0, 1, 2 or 3, where R′″ represents identical or different radicals if n represents 2 or 3,

R³ represents hydrogen, fluorine, chlorine, bromine, iodine, methyl, ethyl, isopropyl, cyclopropyl, cyclobutyl, cyclopentyl, cyclohexyl, trifluoromethyl, 1-trifluoromethyl-2,2,2-trifluoroethyl or heptafluoroisopropyl,

R⁴ represents cyano, fluorine, chlorine, bromine, iodine, nitro, formyl, trifluoromethyl, difluoromethyl, chloromethyl, methyl, ethyl, cyclopropyl, thiocarbamoyl, methoxycarbonyl, methylcarbonyl, ethylcarbonyl, benzylcarbonyl, cyclopropylcarbonyl, cyclopentylcarbonyl, cyclohexylcarbonyl, hydroximinomethyl, methoximinomethyl, methylthio, meihylsulfinyl, methylsulfonyl, methylaminocarbonyl, hydroxymethyl, hydroxyeth-1-yl, methoxymethyl, ethoxymethyl or 1-methoxyethyl,

Hal represents fluorine or chlorine and

R⁵ represents alkyl having 1 to 4 carbon atoms, alkenyl having 2 to 4 carbon atoms, cycloalkyl having 3 to 7 carbon atoms or cycloalkenyl having 3 to 7 carbon atoms, or

R⁵ represents haloalkyl having 1 to 4 carbon atoms and 1 to 5 fluorine, chlorine and/or bromine atoms, haloalkenyl having 3 or 4 carbon atoms and 1 to 3 fluorine, chlorine and/or bromine atoms, cycloalkyl which has 3 to 6 carbon atoms and substituted by 1 to 3 fluorine, chlorine and/or bromine atoms or represents cycloalkenyl which has 3 to 6 carbon atoms and is substituted by 1 to 3 fluorine, chlorine and/or bromine atoms or methyl groups.

A very particularly preferred group of pyrazolopyrimidines according to the invention are those compounds of the formula (I) in which

R¹, R², R⁴ and Hal have the particularly preferred meanings given above,

R³ represents hydrogen, fluorine, chlorine, bromine, methyl, ethyl, propyl, isopropyl, trifluoromethyl or cyclopropyl and

R⁵ represents methyl, ethyl, propyl, isopropyl, n-butyl, i-butyl, sec-butyl, tert-butyl, allyl, but-2-en-1-yl, cyclopropyl, cyclopentyl, 2-methylcyclopentyl, cyclohexyl, 2-methylcyclohexyl, cyclopentenyl, 2-methylcyclopentenyl, 2-chlorocyclopentenyl, cyclohexenyl, 2-methylcyclohexenyl, 2-chlorocyclohexenyl, chloromethyl, trifluoromethyl, trifluoroisopropyl, trichloroallyl, 2,2-dichlorocyclopropyl or dichlorocyclohexenyl.

The radical definitions mentioned above can be combined with one another as desired. Moreover, individual definitions may not apply.

Using 3-cyano-5,7-dichloro-6-(sec-butyl)pyrazolo[1,5-a]pyrimidine and 2,2,2-trifluoroiso-propylamine as starting materials, the course of process (a) according to the invention can be illustrated by the formula scheme below.

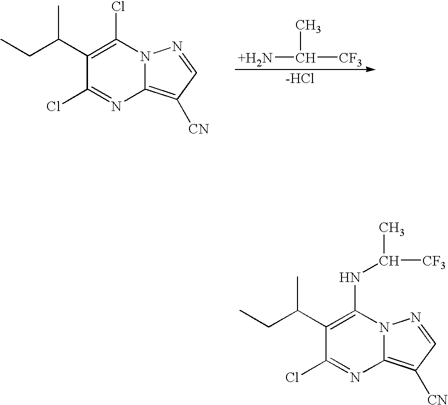

Using 3-cyano-5-chloro-6-(sec-butyl)-7-(2,2,2-trifluoroisopropylamino)pyrazolo[1,5-a]pyrimidine as starting material and diisobutylaluminum hydride as reaction component, the course of the process (b, variant α) according to the invention can be illustrated by the formula scheme below.

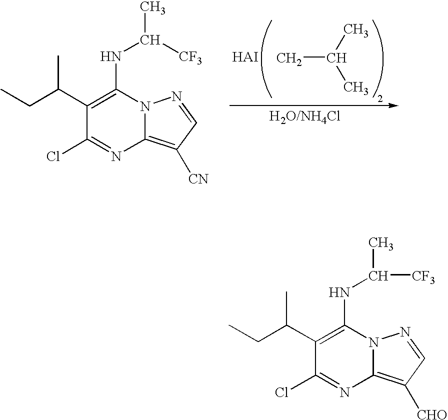

Using 3-cyano-5-chloro-6-(sec-butyl)-7-(2,2,2-trifluoroisopropylamino)pyrazolo[1,5-a]pyrimidine as starting material and methyl magnesium bromide as reaction component, the course of the process (b, variant β) according to the invention can be illustrated by the formula scheme below.

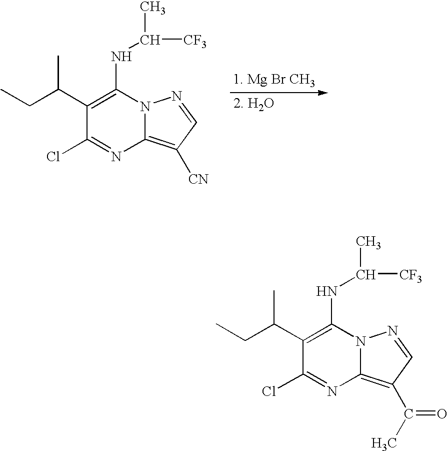

Using 3-formyl-5-chloro-6-(sec-butyl)-7-(2,2,2-trifluoroisopropylamino)pyrazolo[1,5-a]pyrimidine and methoxyamine hydrochloride as starting materials, the course of the process (c, variant α) according to the invention can be illustrated by the formula scheme below.

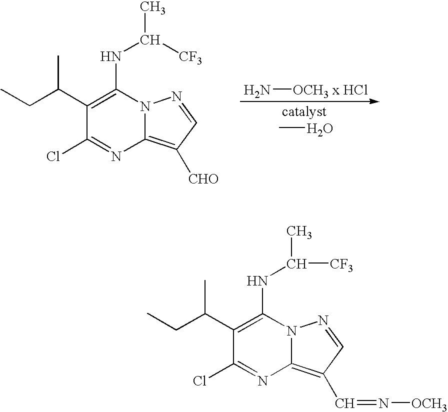

Using 3-methylcarbonyl-5-chloro-6-(sec-butyl)-7-(2,2,2-trifluoroisopropylamino)pyrazolo[1,5-a]-pyrimidine as starting material, diisobutylaluminum hydride as reaction component in the first step and methyl iodide as reaction component in the second step, the course of the process (c, variant β) according to the invention can be illustrated by the formula scheme below.

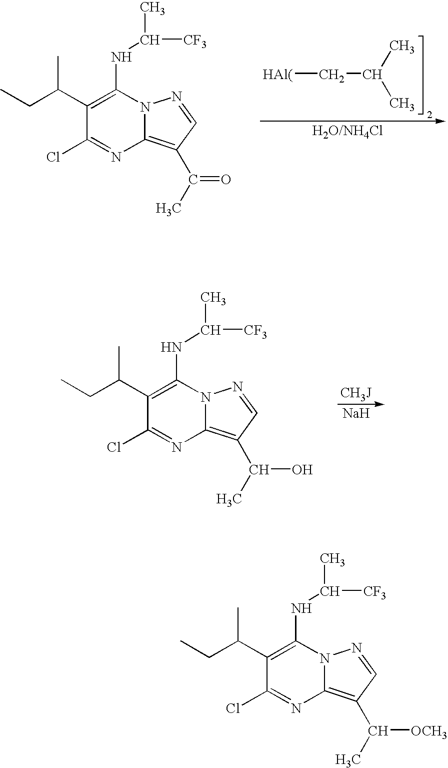

Using 5-chloro-6-(sec-butyl)-7-(2,2,2-trifluoroisopropylamino)pyrazolo[ 1,5-a]pyrimidine and acetyl chloride as starting materials and aluminum trichloride as catalyst, the course of the process (d) according to the invention can be illustrated by the formula scheme below.

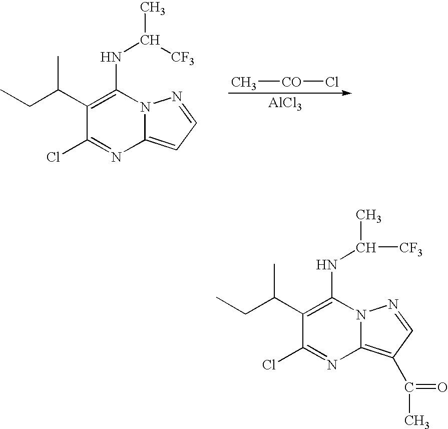

The formula (II) provides a general definition of the halopyrazolopyrimidines required as starting materials for carrying out the process (a) according to the invention. In this formula (II), R³, R⁵ and Hal preferably have those meanings which have already been mentioned in connection with the description of the compounds of the formula (I) according to the invention as being preferred for these radicals.

Y¹ preferably represents fluorine, chlorine or bromine, particularly preferably fluorine or chlorine.

R⁶ preferably represents cyano, fluorine, chlorine, bromine, iodine, nitro, haloalkyl having 1 to 4 carbon atoms and 1 to 9 fluorine, chlorine and/or bromine atoms, alkyl having 1 to 4 carbon atoms, cycloalkyl having 3 to 6 carbon atoms, formyl, thiocarbamoyl, alkoxycarbonyl having 1 to 4 carbon atoms in the alkoxy moiety, alkylthio having 1 to 4 carbon atoms, alkylsulfinyl having 1 to 4 carbon atoms, alkylsulfonyl having 1 to 4 carbon atoms or represents alkylaminocarbonyl having 1 to 4 carbon atoms in the alkyl moiety.

R⁶ particularly preferably represents cyano, fluorine, chlorine, bromine, iodine, nitro, trifluoromethyl, difluoromethyl, methyl, ethyl, cyclopropyl, formyl, thiocarbamoyl, methoxycarbonyl, methylthio, methylsulfinyl, methylsulfonyl or methylaminocarbonyl.

The halopyrazolopyrimidines of the formula (II) can be prepared by
e) reacting hydroxypyrazolopyrimidines of the formula

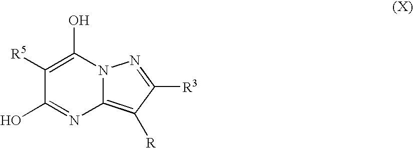

in which
R³ and R⁵ are as defined above
R represents halogen, cyano, nitro, alkyl, haloalkyl, cycloalkyl, thiocarbamoyl, alkoxycarbomyl, alkylthio, alkylsulfinyl, alkylsulfonyl or alkylaminocarbonyl,
with halogenating agents, if appropriate in the presence of a diluent, or
f) reacting hydroxypyrazolopyrimidines of the formula

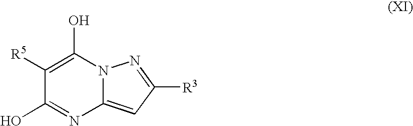

in which
R³ and R⁵ are as defined above,
with phosphorus oxychloride in the presence of dimethylformamide and, if appropriate, allowing to react further with addition of phosphorus pentachloride.

The formula (X) provides a general definition of the hydroxypyrazolopyrimidines required as starting materials for carrying out the process (e). In this formula, R³ and R⁵ preferably have those meanings which have already been mentioned in connection with the description of the compounds of the formula (I) according to the invention as being preferred for these radicals. R preferably represents cyano, fluorine, chlorine, bromine, iodine, nitro, alkyl having 1 to 4 carbon atoms, haloalkyl having 1 to 4 carbon atoms and 1 to 9 fluorine, chlorine, and/or bromine atoms, cycloalkyl having 3 to 6 carbon atoms, thiocarbamoyl, alkylcarbonyl having 1 to 4 carbon atoms in the alkoxy moiety, alkylthio having 1 to 4 carbon atoms, alkylsulfmyl having 1 to 4 carbon atoms, alkylsulfonyl having 1 to 4 carbon atoms or represents alkylaminocarbonyl having 1 to 4 carbon atoms in the alkyl moiety.

R particularly preferably represents cyano, fluorine, chlorine, bromine, iodine, nitro, trifluoromethyl, difluoromethyl, chloromethyl, methyl, ethyl, cyclopropyl, thiocarbamoyl, methoxycarbonyl, methylthio, methylsulfinyl, methylsulfonyl or methylaminocarbonyl.

The hydroxypyrazolopyrimidines of the formula (X) can be prepared by
g) reacting malonic ester derivatives of the formula

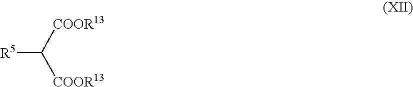

in which
R⁵ is as defined above and
R¹³ represents alkyl having 1 to 4 carbon atoms,
with aminopyrazoles of the formula

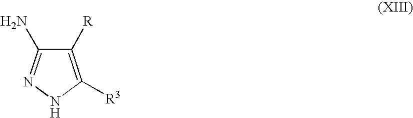

in which
R³ and R are as defined above,
if appropriate in the presence of a diluent and if appropriate in the presence of an acid binder.

The formula (XII) provides a general definition of the malonic ester derivatives required as starting materials for carrying out the process (g). In this formula, R⁵ preferably has those meanings which have already been mentioned in connection with the description of the compounds of the formula (I) according to the invention as being preferred for this radical. R¹³ preferably represents methyl or ethyl.

The malonic ester derivatives of the formula (XII) are known or can be prepared by known methods.

The formula (XIII) provides a general definition of the aminopyrazoles required as reacting components for carrying out the process (g). In this formula, R³ and R preferably have those meanings which have already been mentioned in connection with the description of the compounds of the formula (I) according to the invention or the hydroxypyrazolopyrimidines of the formula (X) as being preferred for these radicals.

The aminopyrazoles of the formula (XIII) are known or can be prepared by known methods.

Suitable diluents for carrying out the process (g) are all inert organic solvents customary for such reactions. Preference is given to using alcohols, such as methanol, ethanol, n-propanol, i-propanol, n-butanol and tert-butanol.

Suitable acid binders for carrying out the process (g) are all inorganic and organic bases customary for such reactions. Preference is given to using tertiary amines, such as tributylamine or pyridine. It is also possible for excess amine to act as diluent.

When carrying out the process (g), the temperatures can be varied within a relatively wide range. In general, the process is carried out at temperatures between 20° C. and 200° C., preferably between 50° C. and 180° C.

When carrying out the process (g), malonic ester derivative of the formula (XII) and aminopyrazole of the formula (XIII) are generally employed in equivalent amounts. However, it is also possible to use an excess of one or the other component. Work-up is carried out with customary methods.

Process (f) is suitable for preparing halopyrazolopyrimidine of the formula

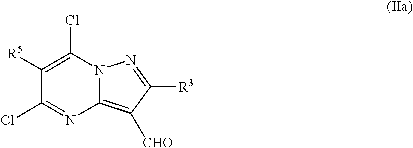

in which

R³ and R⁵ are as defined above.

The formula (XI) provides a general definition of the hydroxypyrazolopyrimidines required as starting materials for carrying out the process (f). In this formula, R³ and R⁵ preferably have those meanings which have already been mentioned in connection with the description of the compounds of the formula (I) according to the invention as being preferred for these radicals.

The hydroxypyrazolopyrimidines of the formula (XI) can be prepared according to process (g).

The process (f) is carried out under the conditions of the Vilsmeier formulation with the aid of phosphorus oxychloride in the presence of dimethylformamide. Here, it is also possible to add phosphorus pentachloride as chlorinating agent.

When carrying out the process (f), the reaction temperatures can be varied within a relatively wide range. In general, the process is carried out at temperatures between −10° C. and +150° C., preferably between 0° C. and 120° C.

When carrying out the process (f), in general from 2 to 5 mol of dimethylformamide, from 5 to 15 mol of phosphorus oxychloride and, if appropriate, from 0 to 2 mol of phosphorus pentachloride are employed per mole of hydroxypyrazolopyrimidine of the formula (XI). Work-up is carried out by customary methods.

Suitable halogenating agents for carrying out the process (e) are all components customary for replacing hydroxyl groups by halogen. Preference is given to using phosphorus trichloride, phosphorus tribromide, phosphorus pentachloride, phosphorus oxychloride, thionyl chloride, thionyl bromide or mixtures thereof. The corresponding fluorine compounds of the formula (II) can be prepared from the chlorine or bromine compounds by reaction with potassium fluoride.

The halogenating agents mentioned are known.

Suitable diluents for carrying out the process (e) are all solvents customary for such halogenations. Preference is given to using halogenated aliphatic or aromatic hydrocarbons, such as chlorobenzene. However, it is also possible for the halogenating agent itself, for example phosphorus oxychloride or a mixture of halogenating agents, to act as diluent.

When carrying out the process (e), the temperatures can also be varied within a relatively wide range. In general, the process is carried out at temperatures between 0° C. and 150° C., preferably between 10° C. and 120° C.

When carrying out the process (e), hydroxypyrazolopyrimidine of the formula (XI) is generally reacted with an excess of halogenating agent. Work-up is carried out by customary methods.

The formula (III) provides a general definition of the amines furthermore required as starting materials for carrying out the process (a). In this formula, R¹ and R² preferably have those meanings which have already been mentioned in connection with the description of the compounds of the formula (I) according to the invention as being preferred for R¹ and R².

The amines of the formula (III) are known or can be prepared by known methods.

Suitable diluents for carrying out the process (a) according to the invention are all customary inert organic solvents. Preference is given to using halogenated hydrocarbons, such as, for example, chlorobenzene, dichlorobenzene, dichloromethane, chloroform, carbon tetrachloride, dichloroethane or trichlorethane; ethers, such as diethyl ether, diisopropyl ether, methyl-t-butyl ether, methyl t-amyl ether, dioxane, tetrahydrofuran, 1,2-dimethoxyethane, 1,2-diethoxyethane or anisole; nitrites, such as acetonitrile, propionitrile, n- or i-butyronitrile or benzonitrile; amides, such as N,N-dimethylformamide, N,N-dimethylacetamide, N-methylformanilide, N-methyl-pyrrolidone or hexamethylphosphoric triamide; esters, such as methyl acetate or ethyl acetate; sulfoxides, such as dimethyl sulfoxide; sulfones, such as sulfolane.

Suitable acidic receptors for carrying out the process (a) according to the invention are all inorganic or organic bases customary for such reactions. Preference is given to using alkali earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate, and furthermore ammonium compounds, such as ammonium hydroxide, ammonium acetate and ammonium carbonate, and also tertiary amines, such as trimethylamine, triethylamine, tributylamine, N,N-dimethylaniline, N,N-dimethylbenzyl-amine, pyridine, N-methylpiperidine, N-methylmorpholine, N,N-dimethylaminoyridine, diazabi-cyclooctane (DABCO), diazabicyclononene (DBN) or diazabicycloundecene (DBU).

Suitable catalysts for carrying out the process (a) according to the invention are all reaction promoters customary for such reactions. Preference is given to using fluorides, such as sodium fluoride, potassium fluoride or ammonium fluoride.

When carrying out the process (a) according to the invention, the reaction temperatures can be varied within a relatively wide range. In general, the process is carried out at temperatures between 0° C. and 150° C., preferably at temperatures between 0° C. and 80° C.

When carrying out the process (a) according to the invention, in general from 0.5 to 10 mol, preferably from 0.8 to 2 mol, of amine of the formula (III) are employed per mole of halopyrazolopyrimidine of the formula (II). Work-up is carried out by customary methods.

The formula (Ia) provides a general definition of the pyrazolopyrimidines required as starting materials for carrying out the process (b) according to the invention. In this formula, R¹, R², R³, R⁵ and Hal preferably have those meanings which have already been mentioned in connection with the description of the compounds of the formula (I) according to the invention as being preferred for these radicals.

The pyrazolopyrimidines of the formula (Ia) are compounds according to the invention which can be prepared by the process (a) according to the invention.

Suitable diluents for carrying out the process (b, variant α) according to the invention are all customary inert inorganic solvents. Preference is given to using aliphatic or aromatic, optionally halogenated hydrocarbons, such as toluene, dichloromethane, chloroform or carbon tetrachloride.

When carrying out the process (b, variant α) according to the invention, the reaction temperatures can be varied within a certain range. In general, the process is carried out at temperatures between −80° C. and +20° C., preferably between −60° C. and +10° C.

When carrying out the process (b, variant α) according to the invention, in general an equivalent amount or else an excess, preferably from 1.1 to 1.2 mol, of diisobutylaluminum hydride is employed per mole of pyrazolopyrimidine of the formula (Ia), and an excess of aqueous ammonium chloride solution is then added. Work-up is carried out by customary methods. In general, the reaction mixture is acidified, the organic phase is removed, the aqueous phase is extracted with a poorly water-miscible organic solvent and the combined organic phases are washed, dried and concentrated under reduced pressure.

The formula (IV) provides a general definition of the Grignard compounds required as reaction components for carrying out the process (b, variant β) according to the invention. In this formula, R⁷ preferably represents alkyl having 1 to 4 carbon atoms, benzyl or cycloalkyl having 3 to 6 carbon atoms. Particularly preferably, R⁷ represents methyl, ethyl, cyclopropyl, cyclopentyl, cyclohexyl or benzyl. X also preferably represents chlorine, bromine or iodine.

Suitable catalysts for carrying out the process (b, variant β) according to the invention are all reaction promoters customary for such Grignard reactions. Potassium iodide and iodine may be mentioned by way of example.

Suitable diluents for carrying out the process (b, variant β) are all inert inorganic solvents customary for such reactions. Preference is given to using ethers, such as diethyl ether, dioxane or tetrahydrofuran, moreover aromatic hydrocarbons, such as toluene, and also mixtures of ethers and aromatic hydrocarbons, such as toluene/tetrahydrofuran.

When carrying out the process (b, variant β), the reaction temperatures can be varied within a certain range. In general, the process is carried out at temperatures between −20° C. and +100° C., preferably between 0° C. and 80° C.

When carrying out the process (b, variant β) according to the invention, in general from 2 to 3 mol of Grignard compound of the formula (IV) are employed per mole of pyrazolopyrimidine of the formula (Ia). This is followed by an aqueous work-up according to customary methods.

The formula (Ib) provides a general definition of the pyrazolopyrimidines required as starting materials for carrying out the process (c) according to the invention. In this formula, R¹, R², R³, R⁵ and Hal preferably have those meanings which have already been mentioned in connection with the description of the compounds of the formula (I) according to the invention as being preferred for these radicals. R⁸ preferably represents hydrogen or alkyl having 1 to 4 carbon atoms, benzyl or cycloalkyl having 3 to 6 carbon atoms. Particularly preferably, R⁵ represents hydrogen methyl, ethyl, benzyl, cyclopropyl, cyclopentyl or cyclohexyl.

The pyrazolopyrimidines of the formula (Ib) are compounds according to the invention which can be prepared by the process (b) according to the invention.

The formula (V) provides a general definition of the amino compounds required as reaction components for carrying out the process (c, variant α) according to the invention. In this formula, R⁹ preferably represents hydrogen or alkyl having 1 to 4 carbon atoms, particularly preferably hydrogen, methyl or ethyl.

Suitable reaction components also include acid addition salts, preferably hydrogen chloride addition salts, of amino compounds of the formula (V).

Both the amino compounds of the formula (V) and their acid addition salts are known or can be prepared by known methods.

Suitable diluents for carrying out the process (c, variant α) according to the invention are all customary inert organic solvents. Preference is given to using alcohols, such as methanol, ethanol, n-propanol or isopropanol.

Suitable catalysts for carrying out the process (c, variant α) according to the invention are all reaction promoters customary for such reactions. Preference is given to using acidic or basic catalysts, such as, for example, weak basic ion exchangers commercially available under the name Amberlyst A-21®.

When carrying out the process (c, variant α), the reaction temperatures can be varied within a certain range. In general, the process is carried out at temperatures between 0° C. and 80° C., preferably between 10° C. and 60° C.

When carrying out the process (c, variant α) according to the invention, in general an equivalent amount or an excess, preferably between 1.1 and 1.5 mol, of amino compound of the formula (V) or an acid addition salt thereof is employed per mole of pyrazolopyrimidine of the formula (Ib). Work-up is carried out by customary methods. In general, the reaction mixture is, if appropriate, filtered and then concentrated and purified.

The formula (VI) provides a general definition of the alkylating agents required as reaction components for carrying out the process (c, variant β) according to the invention. In this formula, R¹⁰ preferably represents alkyl having 1 to 4 carbon atoms, particularly preferably methyl or ethyl, X¹ preferably represents chlorine, bromine, iodine or the radical R¹⁰—O—SO₂—O, in which R¹⁰ is as defined above.

The alkylating agents of the formula (VI) are known or can be prepared by known methods.

If the reducing agent used for carrying out the first step of the process (c, variant β) according to the invention is diisobutylaluminum hydride, the process is expediently carried out under the conditions already mentioned in connection with the description of the process (b, variant α) according to the invention.

If the reducing agent used for carrying out the first step of the process (c, variant β) according to the invention is sodium borohydride, the diluents used are generally alcohols, preferably methanol, ethanol or isopropanol.

In the reduction with sodium borohydride, the reaction temperatures can be varied within a certain range. In general, the process is carried out at temperatures between 0° C. and 70° C., preferably between 0° C. and 50° C.

When carrying out the reduction with sodium borohydride, an equivalent amount or else an excess of sodium borohydride is employed per mole of pyrazolopyrimidine of the formula (Ib). Work-up is again carried out by customary methods.

Suitable bases for carrying out the second step of the process (c, variant β) according to the invention are all customary acid binders. Preference is given to using alkali metal hydrides, alkoxides and carbonates, such as sodium hydride, sodium methoxide, potassium tert-butoxide, sodium carbonate, potassium carbonate or lithium carbonate.

Suitable diluents for carrying out the second step of the process (c, variant β) according to the invention are all customary inert organic solvents. Preference is given to using ethers, such as dioxane or tetrahydrofuran, and furthermore nitrites, such as acetonitrile.

When carrying out the second step of the process (c, variant β) according to the invention, the temperatures can be varied within a relatively wide range. In general, the process is carried out at temperatures between 0° C. and 100° C., preferably between 20° C. and 80° C.

When carrying out the second step of the process (c, variant β) according to the invention, in general from 1 to 2 mol, preferably from 1 to 1.5 mol, of alkylinating agent of the formula (VI) are employed per mole of pyrazolopyrimidine of the formula (Ic). Work-up is again carried out by customary methods.

The formula (VII) provides a general definition of the pyrazolopyrimidines required as starting materials for carrying out the process (d) according to the invention. In this formula, R¹, R², R³, R⁵ and Hal preferably have those meanings which have already been mentioned in connection with the description of the compounds of the formula (I) according to the invention as being preferred for these radicals.

The pyrazolopyrimidines of the formula (VII) are known or can be prepared by known methods.

The formulae (VIII) and (IX) provide general definitions of the acid halides and acid anhydrides required as reaction components for carrying out the process (d) according to the invention. In the formula (VIII), R¹¹ preferably represents alkyl having 1 to 4 carbon atoms, benzyl or cycloalkyl having 3 to 6 carbon atoms. X² preferably represents chlorine or bromine.

Particular preference is given to acid halides of the formula (VIII) in which

R¹¹ represents methyl, ethyl, propyl, benzyl, cyclopropyl, cyclopentyl or cyclohexyl and

X² represents chlorine or bromine.

In the formula (IX), R¹² preferably represents alkyl having 1 to 4 carbon atoms, particularly preferably methyl, ethyl or propyl.

Both the acid halides of the formula (VIII) and the acid anhydrides of the formula (IX) are known or can be prepared by known methods.

Suitable catalysts for carrying out the process (d) according to the invention are all reaction promoters which are customarily used for Friedel-Crafts reactions. Preference is given to using Lewis acids, such as aluminum trichloride, aluminum tribromide and iron(III) chloride.

Suitable diluents for carrying out the process (d) according to the invention are all inert organic solvents customary for such Friedel-Crafts reactions. Preference is given to using ethers, such as diethyl ether, methyl tert-butyl ether, dioxane and tetrahydrofuran, and also carbon disulfide.

When carrying out the process (d) according to the invention, the reaction temperatures can be varied within a certain range. In general, the process is carried out at temperatures between −10° C. and +100° C., preferably between 0° C. and 80° C.

When carrying out the process (d) according to the invention, in general from 1 to 5 mol, preferably from 1 to 2 mol, of acid halide of the formula (VIII) and from 1.1 to 5 mol, preferably from 1.1 to 3 mol, of catalyst or from 1 to 5 mol, preferably from 1 to 2 mol, of acid anhydride of the formula (IX) and from 2.1 to 6 mol, preferably from 2.1 to 4 mol, of catalyst are employed per mole of pyrazolopyrimidine of the formula (VII). In general, the reaction components are initially added at low temperature and, after the initially vigorous reaction has subsided, slowly heated to reflux temperature. Work-up is carried out by customary methods.

All of the processes described above are generally carried out under atmospheric pressure. However, it is also possible to operate under elevated pressure.

The compounds according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:
Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such as, for example, Pythium ultimum;
Phytophthora species, such as, for example, Phytophthora infestans;
Pseudoperonospora species, such as, for example, Pseudoperonospora humuli or Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea (conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasakii;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and
Pseudocercosporella species, such as, for example, Pseudocercosporella herpotrichoides.

The active compounds according to the invention also show a strong invigorating action in plants. Accordingly, they are suitable for mobilizing the internal defenses of the plant against attack by unwanted microorganisms.

In the present context, plant-invigorating (resistance-inducing) compounds are to be understood as meaning substances which are capable of stimulating the defense system of plants such that, when the treated plants are subsequently inoculated with unwanted microorganisms, they display substantial resistance to these microorganisms.

In the present case, unwanted microorganisms are to be understood as meaning phytopathogenic fungi, bacteria and viruses. The compounds according to the invention can thus be used to protect plants within a certain period of time after treatment against attack by the pathogens mentioned. The period of time for which this protection is achieved generally extends for 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed with particularly good results for controlling cereal diseases, such as, for example, against Erysiphe species, and of diseases in viticulture and in the cultivation of fruit and vegetables, such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates and precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes.

Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular molds, wood-discoloring and wood-destroying fungi (Basidiomycetes) and against slime organisms and algae.

Microorganisms of the following genera may be mentioned as examples:
Altemaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene. chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulfoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as fmely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, or else protein hydrolyzates. Suitable dispersants are: for example lignosulfite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 per cent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

Fungicides:

2-phenylphenol; 8-hydroxyquinoline sulfate; acibenzolar-S-methyl; aldimorph; amidoflumet; ampropylfos; ampropylfos-potassium; andoprim; anilazine; azaconazole; azoxystrobin; benalaxyl; benalaxyl-M, benodanil; benomyl; benthiavalicarb-isopropyl; benzamacril; benzamacril-isobutyl; bilanafos; binapacryl; biphenyl; bitertanol; blasticidin-S; boscalid; bromuconazole; bupirimate; buthiobate; butylamine; calcium polysulfide; capsimycin; captafol; captan; carbendazim; carboxin; carpropamid; carvone; chinomethionat; chlobenthiazone; chlorofenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymi-done; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrin; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridine-dicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxa-spiro[4.5]decane-3-amine; sodium tetracarbonate;

and copper salts and preparations, such as Bordeaux mixture; copper hydroxide; copper naphthenate; copper oxychloride; copper sulfate; cufraneb; copper oxide; mancopper; oxinecopper.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/Acaricides/Nematicides:

1. Acetylcholinesterase (AChE) Inhibitors

1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)

1.2 organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulfone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl 0-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

2. Sodium Channel Modulators/Blockers of Voltage-Gated Sodium Channels

2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (lR-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (lR-isomer), tralomethrin, transfluthrin, ZXI 8901, pyrethrins (pyrethrum))

2.2 oxadiazines (for example indoxacarb)

3. Acetylcholine Receptor Agonists/Antagonists

3.1 chloronicotinyls/neonicotinoids (for example acetamiprid, clothianidin, dinotefuran, imidacloprid, nitenpyram, nithiazine, thiacloprid, thiamethoxam)

3.2 nicotines, bensultap, cartap

4. Acetylcholine Receptor Modulators

4.1 spinosyns (for example spinosad)

5. Antagonists of GABA-Gated Chloride Channels

5.1 cyclodiene organochlorines (for example camphechlor, chlordane, endosulfan, gamma-HCH, HCH, heptachlor, lindane, methoxychlor

5.2 fiproles (for example acetoprole, ethiprole, fipronil, vaniliprole)

6. Chloride Channel Activators

6.1 mectins (for example abamectin, avermectin, emamectin, emamectin-benzoate, ivermectin, milbemectin, milbemycin)

7. Juvenile Hormone Mimetics

(for example diofenolan, epofenonane, fenoxycarb, hydroprene, kinoprene, methoprene, pyriproxifen, triprene)

8. Ecdyson Agonists/Disruptors

8.1 diacylhydrazines (for example chromafenozide, halofenozide, methoxyfenozide, tebufenozide)

9. Chitin Biosynthesis Inhibitors

9.1 benzoylureas (for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron)

9.2 buprofezin

9.3 cyromazine

10. Inhibitors of Oxidative Phosphorylation, ATP Disruptors

10.1 diafenthiuron

10.2 organotins (for example azocyclotin, cyhexatin, fenbutatin-oxide)

11. Decouplers of Oxidative Phosphorylation Acting by Interrupting the H-Proton Gradient

11.1 pyrroles (for example chlorfenapyr)

11.2 dinitrophenols (for example binapacryl, dinobuton, dinocap, DNOC)

12. Site-I Electron Transport Inhibitors

12.1 METIs (for example fenazaquin, fenpyroximate, pyrimidifen, pyridaben, tebufenpyrad, tolfenpyrad)

12.2 hydramethylnone

12.3 dicofol

13. Site-II Electron Transport Inhibitors

13.1 rotenone

14. Site-III Electron Transport Inhibitors

14.1 acequinocyl, fluacrypyrim

15. Microbial Disruptors of the Insect Gut Membrane

Bacillus Thuringiensis Strains

16. Inhibitors Offat Synthesis

16.1 tetronic acids (for example spirodiclofen, spiromesifen)

16.2 tetramic acids [for example 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg. No.: 203313-25-1)]

17. Carboxamides

(for example flonicamid)

18. Octopaminergic Agonists

(for example amitraz)

19. Inhibitors of Magnesium-Stimulated ATPase

(for example propargite)

20. Phthalamides

(for example N²-[1,1-dimethyl-2-(methylsulfonyl)ethyl]-3-iodo-N′-[2-methyl-4-[1,2,2,2-tetrafluoro-l-(trifluoromethyl)ethyl]phenyl]-1,2-benzenedicarboxamide (CAS Reg. No.: 272451-65-7), flubendiamide)

21. Nereistoxin Analog

(for example thiocyclam hydrogen oxalate, thiosultap-sodium)

22. Biologicals, Hormones or Pheromones

(for example azadirachtin, Bacillus spec., Beauveria spec., codlemone, Metarrhizium spec., Paecilomyces spec., thuringiensin, Verticillium spec.)

23. Active Compounds with Unknown or Unspecific Mechanisms of Action

23.1 fumigants (for example aluminum phosphide, methyl bromide, sulfuryl fluoride)

23.2 selective antifeedants (for example cryolite, flonicamid, pymetrozine)

23.3 mite growth inhibitors (for example clofentezine, etoxazole, hexythiazox)

23.4 amidoflumet, benclothiaz, benzoximate, bifenazate, bromopropylate, buprofezin, chinomethionat, chlordimeform, chlorbenzilate, chlorpicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin,

furthermore the compound 3-methylphenyl propylcarbamate (Tsumacide Z), the compound 3{5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

In addition, the compounds of the formula (I) according to the invention also have very good antimycotic activity. They have a very broad antimycotic activity spectrum in particular against dermatophytes and yeasts, molds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10 000 g/ha, preferably between 1 and 5 000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn, soybeans, potatoes, cotton, tobacco, oilseed rape and also fruit plants (with the fruits apples, pears, citrus fruits and grapes), and particular emphasis is given to corn, soybeans, potatoes, cotton, tobacco and oilseed rape. Traits that are particularly emphasized are increased defense of the plants against insects, arachnids, nematodes and slugs and snails by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defense of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are corn varieties, cotton varieties, soybean varieties and potato varieties which are sold under the trade names YIELD GARD® (for example corn, cotton, soybeans), KnockOut® (for example corn), StarLink® (for example corn), Bollgard® (cotton), Nucoton® (cotton) and NewLeaf® (potato). Examples of herbicide-tolerant plants which may be mentioned are corn varieties, cotton varieties and soybean varieties which are sold under the trade names Roundup Ready® (tolerance to glyphosate, for example corn, cotton, soy bean), Liberty Link® (tolerance to phosphinotricin, for example oilseed rape), IMI® (tolerance to imidazolinones) and STS® (tolerance to sulfonylureas, for example corn). Herbicide-resistant plants (plants bred in a conventional manner for herbicide tolerance) which may be mentioned also include the varieties sold under the name Clearfield® (for example corn). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preferred ranges stated above for the active compounds or mixtures also apply to the treatment of these plants. Particular emphasis is given to the treatment of plants with the compounds or mixtures specifically mentioned in the present text.

The compounds of the formula (I) according to the invention are furthermore suitable for suppressing the growth of tumor cells in humans and mammals. This is based on an interaction of the compounds according to the invention with tubulin and microtubuli and by promoting microtubuli polymerization.

For this purpose, it is possible to administer an effective amount of one or more compounds of the formula (I) or pharmaceutically acceptable salts thereof.

The preparation and the use of the active compounds according to the invention is illustrated in the examples below.

### PREPARATION EXAMPLES

### Example 1

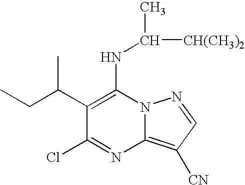

Process (a):

At 0° C., 1.0 g (0.004 mol) of 3-cyano-5,7-dichloro-6-(sec-butyl)pyrazolo[1,5-a]pyrimidine is added with stirring to a solution of 0.389 g (0.004 mol) of (S+)-3-methyl-2-butylamine in 0.451 g (0.004 mol) of triethylamine and 20 ml of dichlorethane. The reaction mixture is stirred at room temperature for 16 hours and then, with stirring, poured into water. The resulting mixture is acidified by addition of hydrochloric acid and extracted with dichlormethane. The combined organic phases are dried over sodium sulfate and then concentrated under reduced pressure. The residue that remains is chromatographed on silica gel using cyclohexane:ethyl acetate =8:2. This gives 0.8 g (64.3% of theory) of 3-cyano-5-chloro-6-(sec-butyl)-7-[(S+)-3-methyl-2-butylamino]pyrazolo[1,5-a]pyrimidine.

HPLC: logP=4.59

### Example 2

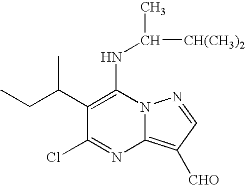

Process (a):

At room temperature, 0.51 g of potassium carbonate and 0.32 g of 3-methyl-2-butylamine are added successively with stirring to a mixture of 1 g of 3-formyl-5,7-dichloro-6-(sec-butyl)-pyrazolo[1,5-a]pyrimidine and 30 ml of acetonitrile. The reaction mixture is stirred at room temperature for 12 hours and then, with stirring, poured into water. The resulting mixture is extracted three times with ethyl acetate. The combined organic phases are dried over sodium sulfate and then concentrated under reduced pressure. The residue that remains is chromatographed on silica gel using cyclohexane:ethyl acetate=4.1. This gives 0.14 g (9.2% of theory) of 3-formyl-5-chloro-6-(sec-butyl)-7-(3-methyl-2-butylamino)pyrazolo[1,5-a]pyrimidine.

HPLC: logP=4.17

The pyrazolopyrimidines of the formula

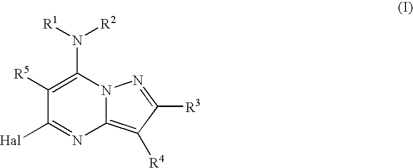

listed in table 1 below are/were also obtained by the methods described above.

TABLE 1

| Ex.No. | 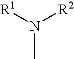 | R³ | R⁴ | Hal | R⁵ | logP *) |
|---|---|---|---|---|---|---|
| 3 | 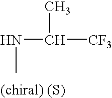 | H | —CN | Cl | 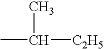 | 3.90 |
| 4 | 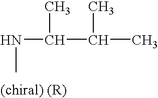 | H | —CN | Cl | 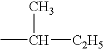 | 4.62 |
| 5 | 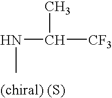 | H | —COOCH₃ | Cl | 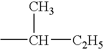 | 3.76 |
| 6 | 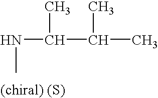 | H | —COOCH₃ | Cl | 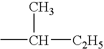 | 4.49 |
| 7 | 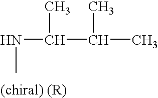 | H | —COOCH₃ | Cl | 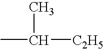 | 4.49 |
| 8 | 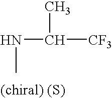 | H | 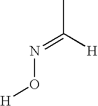 | Cl | 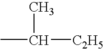 | |
| 9 | 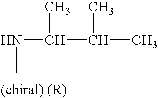 | H | 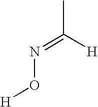 | Cl | 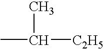 | |
| 10 | 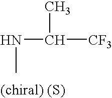 | H | 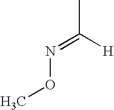 | Cl | 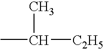 | |
| 11 | 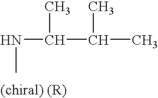 | H | 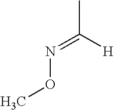 | Cl | 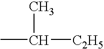 | |
| 12 | 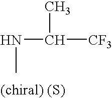 | H | 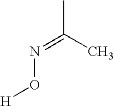 | Cl | 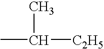 | |
| 13 | 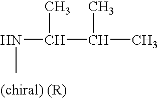 | H | 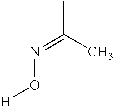 | Cl | 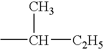 | |
| 14 | 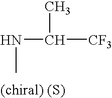 | H | 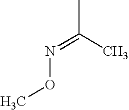 | Cl | 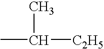 | |
| 15 | 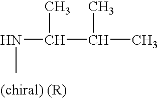 | H | 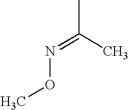 | Cl | 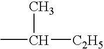 | |
| 16 | 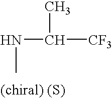 | H | —CN | Cl | 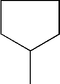 | |
| 17 | 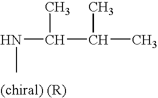 | H | —CN | Cl | 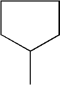 | |
| 18 | 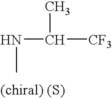 | H | 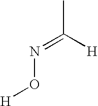 | Cl | 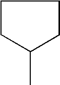 | |
| 19 | 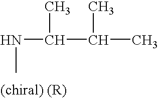 | H | 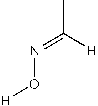 | Cl | 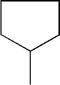 | |
| 20 | 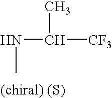 | H | 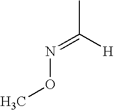 | Cl | 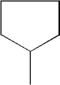 | |
| 21 | 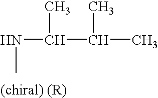 | H | 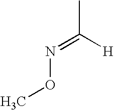 | Cl | 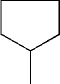 | |
| 22 | 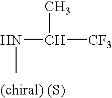 | H | 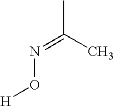 | Cl | 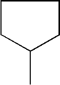 | |
| 23 | 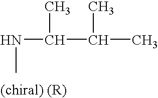 | H | 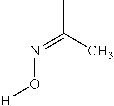 | Cl | 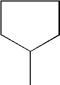 | |
| 24 | 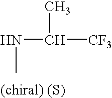 | H | 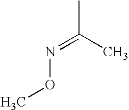 | Cl | 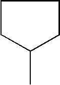 | |
| 25 | 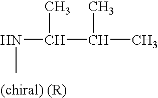 | H | 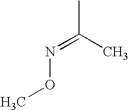 | Cl | 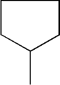 | |
| 26 | 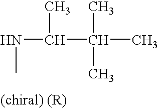 | H | 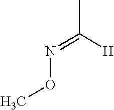 | Cl | 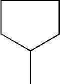 | |
| 27 | 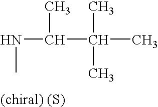 | H | 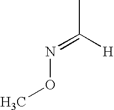 | Cl | 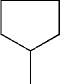 | |
| 28 | 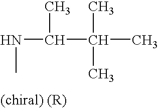 | H | 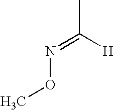 | Cl | 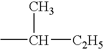 | |
| 29 | 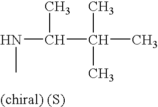 | H | 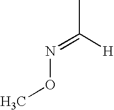 | Cl | 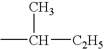 | |
| 34 | 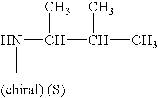 | H | —CN | Cl | 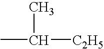 | |
| 35 | 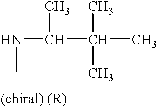 | H | —CN | Cl | 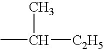 | |
| 36 | 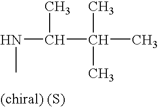 | H | —CN | Cl | 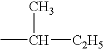 | |
| 37 | 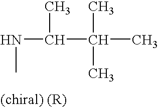 | H | —COOCH₃ | Cl | 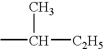 | |
| 38 | 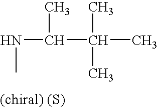 | H | —COOCH₃ | Cl | 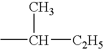 | |
| 39 | 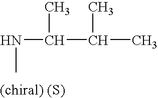 | H | 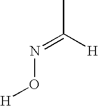 | Cl | 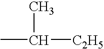 | |
| 40 | 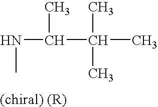 | H | 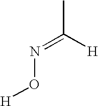 | Cl | 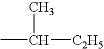 | |
| 41 | 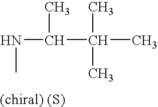 | H | 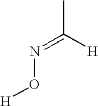 | Cl | 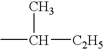 | |
| 42 | 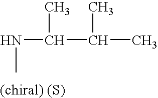 | H | 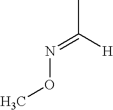 | Cl | 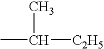 | |
| 43 | 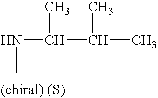 | H | 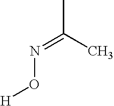 | Cl | 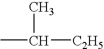 | |
| 44 | 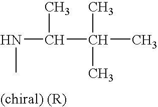 | H | 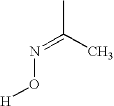 | Cl | 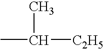 | |
| 45 | 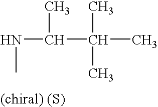 | H | 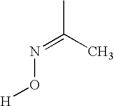 | Cl | 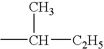 | |
| 46 | 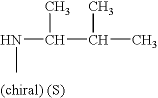 | H | 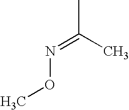 | Cl | 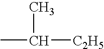 | |
| 47 | 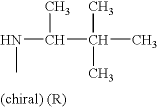 | H | 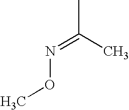 | Cl | 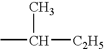 | |
| 48 | 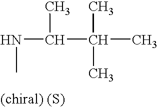 | H | 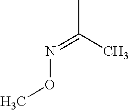 | Cl | 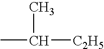 | |
| 49 | 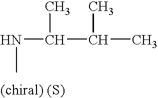 | H | —CN | Cl | 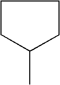 | |
| 50 | 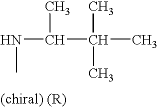 | H | —CN | Cl | 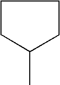 | |
| 51 | 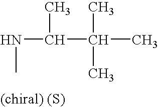 | H | —CN | Cl | 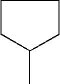 | |
| 52 | 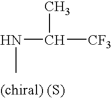 | H | —COOCH₃ | Cl | 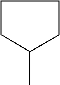 | |
| 53 | 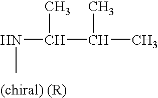 | H | —COOCH₃ | Cl | 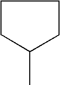 | |
| 54 | 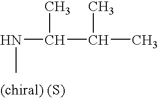 | H | —COOCH₃ | Cl | 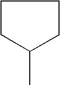 | |
| 55 | 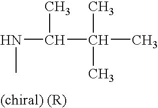 | H | —COOCH₃ | Cl | 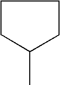 | |
| 56 | 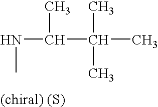 | H | —COOCH₃ | Cl | 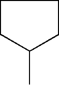 | |
| 57 | 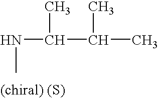 | H | 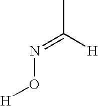 | Cl | 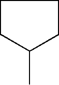 | |
| 58 | 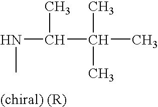 | H | 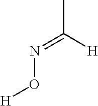 | Cl | 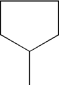 | |
| 59 | 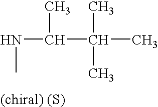 | H | 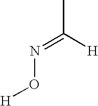 | Cl | 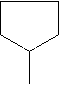 | |
| 60 | 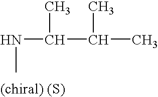 | H | 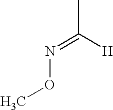 | Cl | 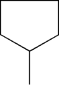 | |
| 61 | 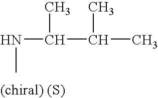 | H | 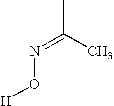 | Cl | 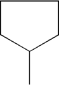 | |
| 62 | 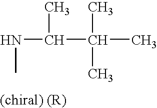 | H | 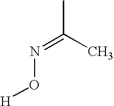 | Cl | 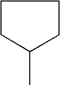 | |
| 63 | 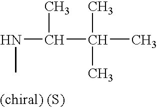 | H | 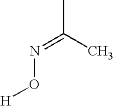 | Cl | 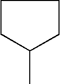 | |
| 64 | 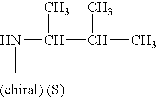 | H | 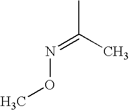 | Cl | 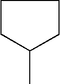 | |
| 65 | 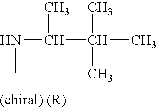 | H | 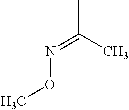 | Cl | 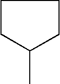 | |
| 66 | 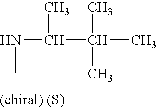 | H | 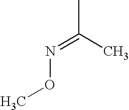 | Cl | 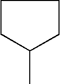 | |
| 67 | 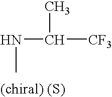 | H | —CN | Cl | 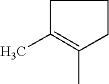 | |
| 68 | 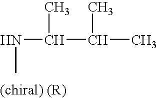 | H | —CN | Cl | 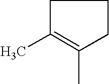 | |
| 69 | 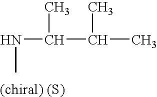 | H | —CN | Cl | 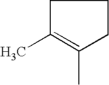 | |
| 70 | 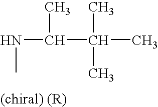 | H | —CN | Cl | 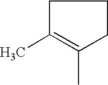 | |
| 71 | 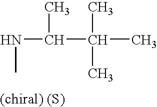 | H | —CN | Cl | 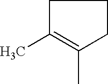 | |
| 72 | 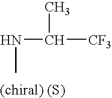 | H | —COOCH₃ | Cl | 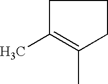 | |
| 73 | 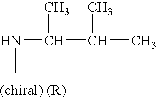 | H | —COOCH₃ | Cl | 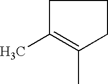 | |
| 74 | 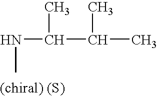 | H | —COOCH₃ | Cl | 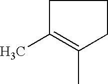 | |
| 75 | 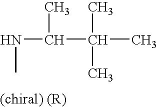 | H | —COOCH₃ | Cl | 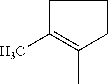 | |
| 76 | 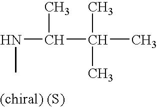 | H | —COOCH₃ | Cl | 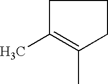 | |
| 77 | 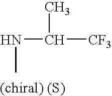 | H | 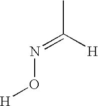 | Cl | 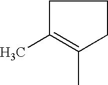 | |
| 78 | 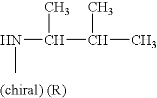 | H | 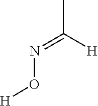 | Cl | 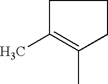 | |
| 79 | 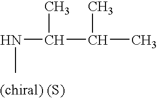 | H | 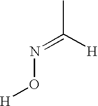 | Cl | 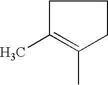 | |
| 80 | 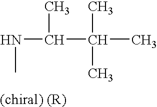 | H | 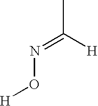 | Cl | 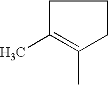 | |
| 81 | 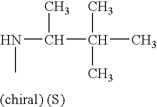 | H | 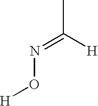 | Cl | 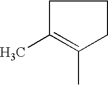 | |
| 82 | 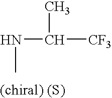 | H | 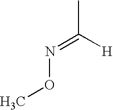 | Cl | 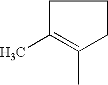 | |
| 83 | 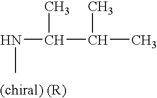 | H | 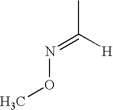 | Cl | 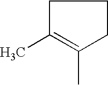 | |
| 84 | 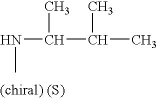 | H | 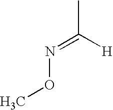 | Cl | 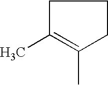 | |
| 85 | 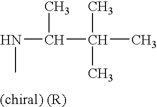 | H | 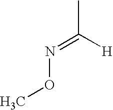 | Cl | 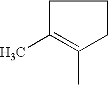 | |
| 86 | 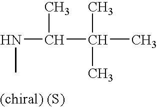 | H | 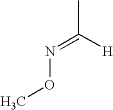 | Cl | 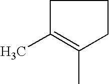 | |
| 87 | 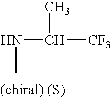 | H | 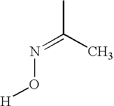 | Cl | 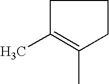 | |
| 88 | 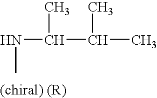 | H | 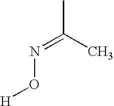 | Cl | 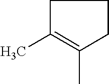 | |
| 89 | 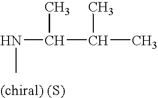 | H | 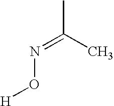 | Cl | 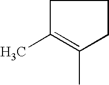 | |
| 90 | 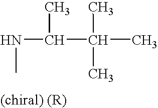 | H | 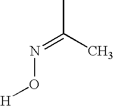 | Cl | 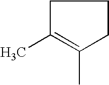 | |
| 91 | 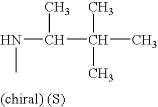 | H | 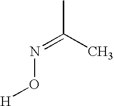 | Cl | 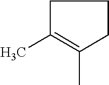 | |
| 92 | 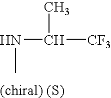 | H | 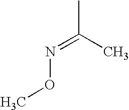 | Cl | 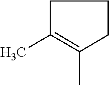 | |
| 93 | 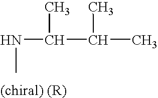 | H | 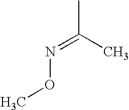 | Cl | 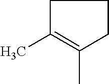 | |
| 94 | 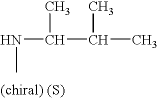 | H | 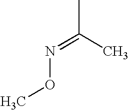 | Cl | 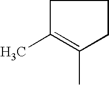 | |
| 95 | 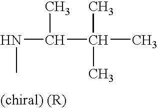 | H | 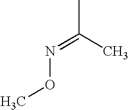 | Cl | 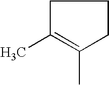 | |
| 96 | 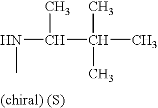 | H | 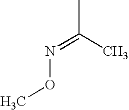 | Cl | 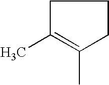 | |
| 97 | 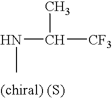 | H | —CN | Cl | 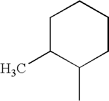 | |
| 98 | 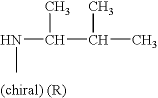 | H | —CN | Cl | 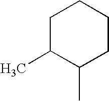 | |
| 99 | 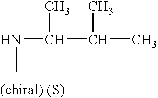 | H | —CN | Cl | 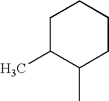 | |
| 100 | 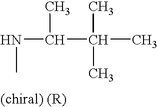 | H | —CN | Cl | 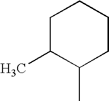 | |
| 101 | 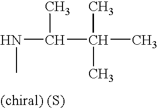 | H | —CN | Cl | 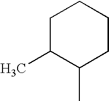 | |
| 102 | 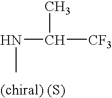 | H | —COOCH₃ | Cl | 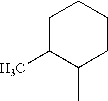 | |
| 103 | 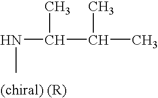 | H | —COOCH₃ | Cl | 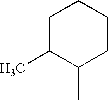 | |
| 104 | 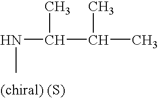 | H | —COOCH₃ | Cl | 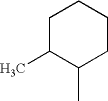 | |
| 105 | 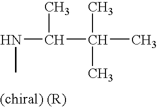 | H | —COOCH₃ | Cl | 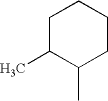 | |
| 106 | 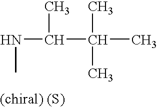 | H | —COOCH₃ | Cl | 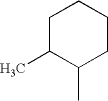 | |
| 107 | 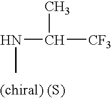 | H | 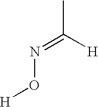 | Cl | 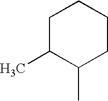 | |
| 108 | 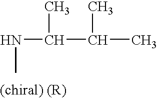 | H | 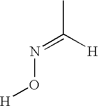 | Cl | 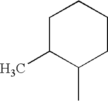 | |
| 109 | 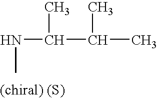 | H | 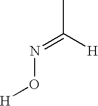 | Cl | 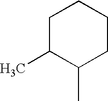 | |
| 110 | 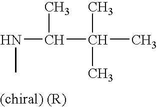 | H | 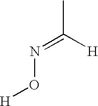 | Cl | 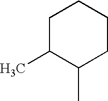 | |
| 111 | 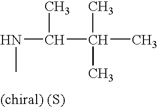 | H | 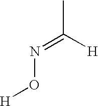 | Cl | 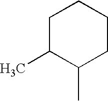 | |
| 112 | 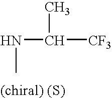 | H | 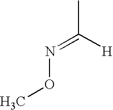 | Cl | 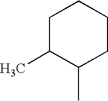 | |
| 113 | 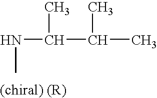 | H | 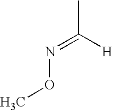 | Cl | 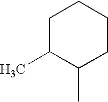 | |
| 114 | 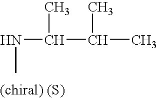 | H | 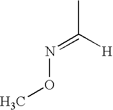 | Cl | 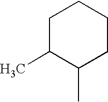 | |
| 115 | 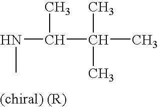 | H | 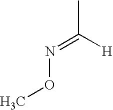 | Cl | 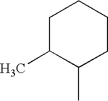 | |
| 116 | 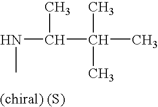 | H | 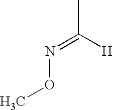 | Cl | 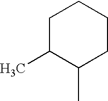 | |
| 117 | 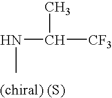 | H | 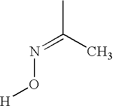 | Cl | 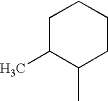 | |
| 118 | 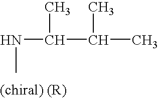 | H | 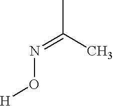 | Cl | 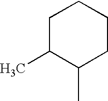 | |
| 119 | 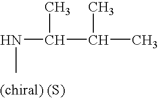 | H | 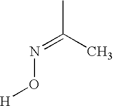 | Cl | 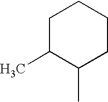 | |
| 120 | 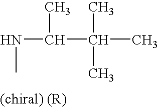 | H | 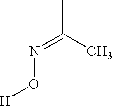 | Cl | 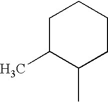 | |
| 121 | 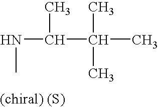 | H | 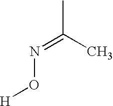 | Cl | 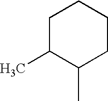 | |
| 122 | 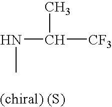 | H | 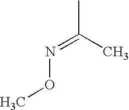 | Cl | 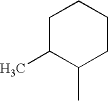 | |
| 123 | 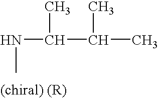 | H | 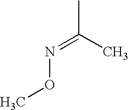 | Cl | 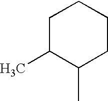 | |
| 124 | 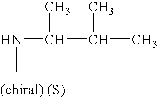 | H | 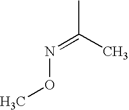 | Cl | 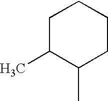 | |
| 125 | 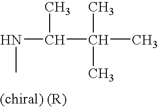 | H | 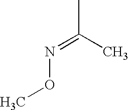 | Cl | 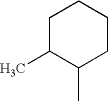 | |
| 126 | 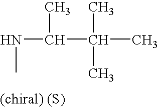 | H | 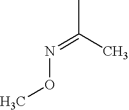 | Cl | 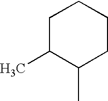 | |
| 127 | 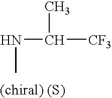 | H | —CN | Cl | 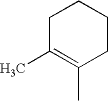 | |
| 128 | 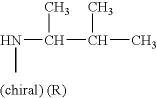 | H | —CN | Cl | 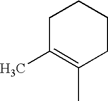 | |
| 129 | 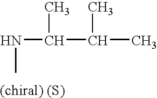 | H | —CN | Cl | 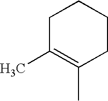 | |
| 130 | 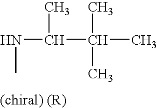 | H | —CN | Cl | 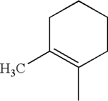 | |
| 131 | 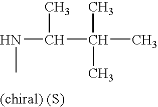 | H | —CN | Cl | 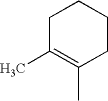 | |
| 132 | 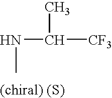 | H | —COOCH₃ | Cl | 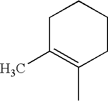 | |
| 133 | 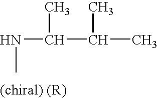 | H | —COOCH₃ | Cl | 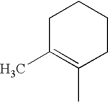 | |
| 134 | 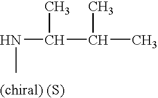 | H | —COOCH₃ | Cl | 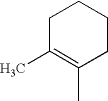 | |
| 135 | 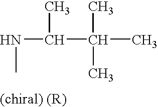 | H | —COOCH₃ | Cl | 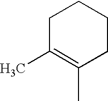 | |
| 136 | 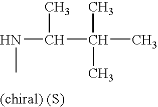 | H | —COOCH₃ | Cl | 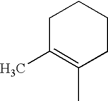 | |
| 137 | 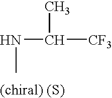 | H | 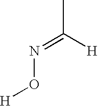 | Cl | 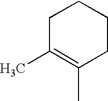 | |
| 138 | 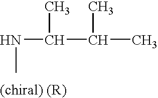 | H | 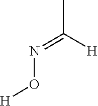 | Cl | 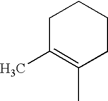 | |
| 139 | 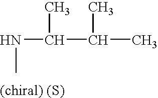 | H | 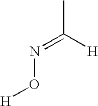 | Cl | 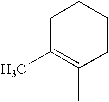 | |
| 140 | 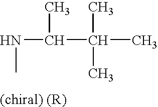 | H | 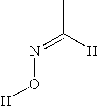 | Cl | 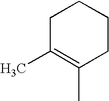 | |
| 141 | 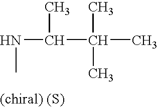 | H | 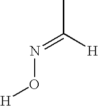 | Cl | 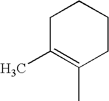 | |
| 142 | 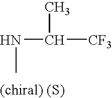 | H | 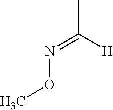 | Cl | 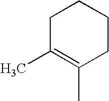 | |
| 143 | 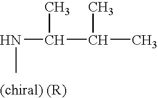 | H | 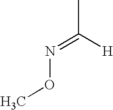 | Cl | 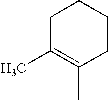 | |
| 144 | 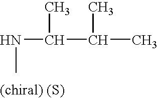 | H | 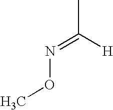 | Cl | 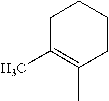 | |
| 145 | 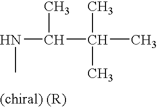 | H | 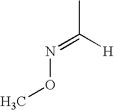 | Cl | 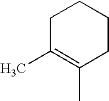 | |
| 146 | 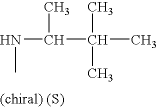 | H | 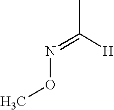 | Cl | 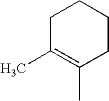 | |
| 147 | 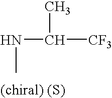 | H | 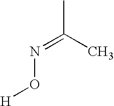 | Cl | 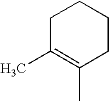 | |
| 148 | 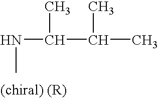 | H | 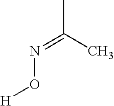 | Cl | 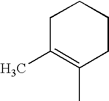 | |
| 149 | 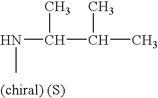 | H | 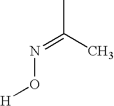 | Cl | 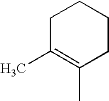 | |
| 150 | 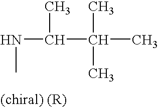 | H | 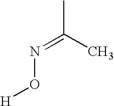 | Cl | 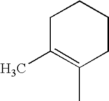 | |
| 151 | 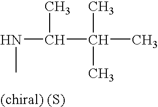 | H | 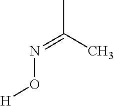 | Cl | 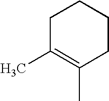 | |
| 152 | 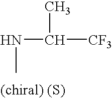 | H | 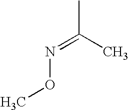 | Cl | 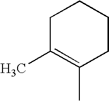 | |
| 153 | 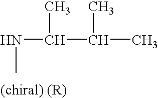 | H | 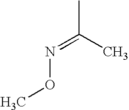 | Cl | 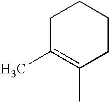 | |
| 154 | 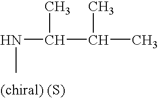 | H | 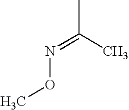 | Cl | 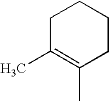 | |
| 155 | 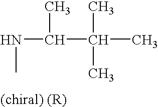 | H | 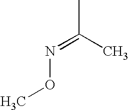 | Cl | 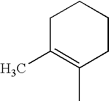 | |
| 156 | 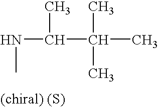 | H | 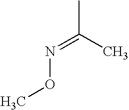 | Cl | 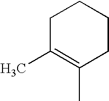 | |
| 157 | 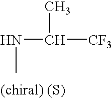 | H | —CN | Cl | 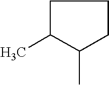 | |
| 158 | 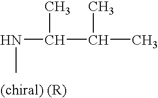 | H | —CN | Cl | 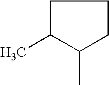 | |
| 159 | 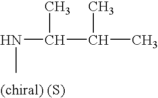 | H | —CN | Cl | 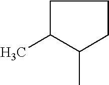 | |
| 160 | 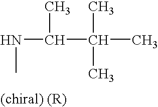 | H | —CN | Cl | 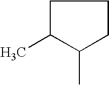 | |
| 161 | 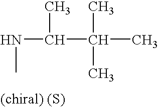 | H | —CN | Cl | 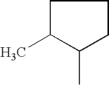 | |
| 162 | 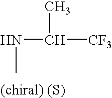 | H | —COOCH₃ | Cl | 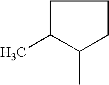 | |
| 163 | 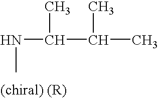 | H | —COOCH₃ | Cl | 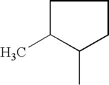 | |
| 164 | 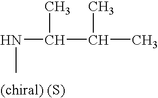 | H | —COOCH₃ | Cl | 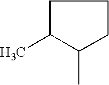 | |
| 165 | 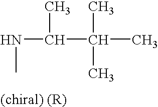 | H | —COOCH₃ | Cl | 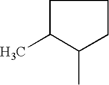 | |
| 166 | 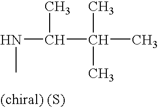 | H | —COOCH₃ | Cl | 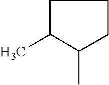 | |
| 167 | 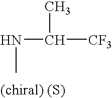 | H | 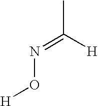 | Cl | 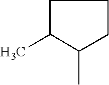 | |
| 168 | 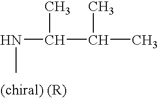 | H | 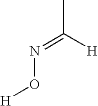 | Cl | 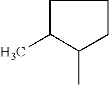 | |
| 169 | 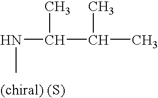 | H | 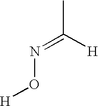 | Cl | 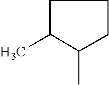 | |
| 170 | 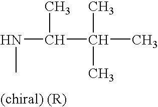 | H | 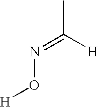 | Cl | 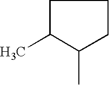 | |
| 171 | 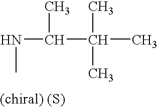 | H | 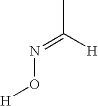 | Cl | 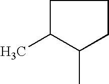 | |
| 172 | 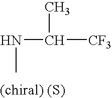 | H | 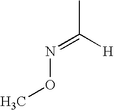 | Cl | 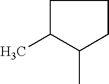 | |
| 173 | 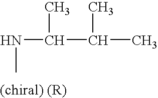 | H | 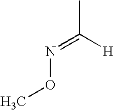 | Cl | 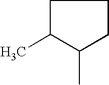 | |
| 174 | 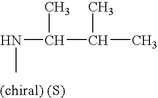 | H | 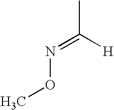 | Cl | 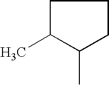 | |
| 175 | 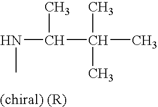 | H | 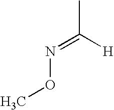 | Cl | 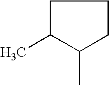 | |
| 176 | 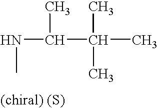 | H | 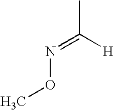 | Cl | 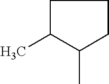 | |
| 177 | 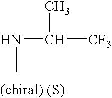 | H | 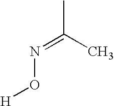 | Cl | 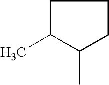 | |
| 178 | 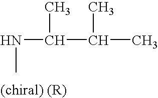 | H | 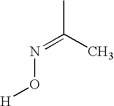 | Cl | 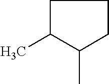 | |
| 179 | 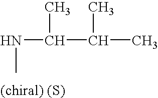 | H | 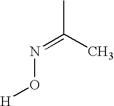 | Cl | 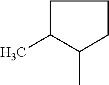 | |
| 180 | 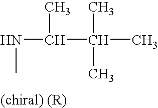 | H | 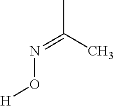 | Cl | 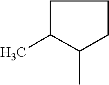 | |
| 181 | 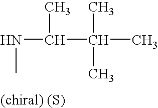 | H | 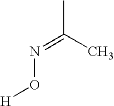 | Cl | 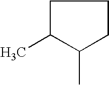 | |
| 182 | 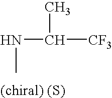 | H | 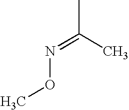 | Cl | 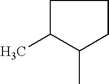 | |
| 183 | 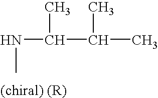 | H | 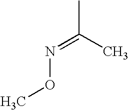 | Cl | 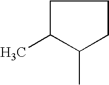 | |
| 184 | 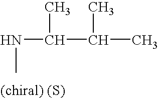 | H | 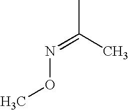 | Cl | 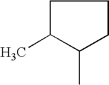 | |
| 185 | 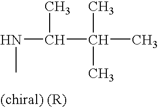 | H | 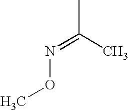 | Cl | 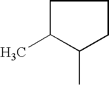 | |
| 186 | 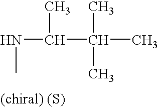 | H | 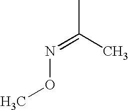 | Cl | 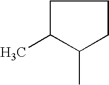 | |

*) logP values were determined in accordance with EEC Directive 79/831 Annex V. A8 by HPLC (gradient method, acetonitrile/0.1% aqueous phosphoric acid)

### Preparation of Starting Materials

### Example 30

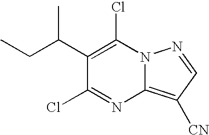

Process (e):

At room temperature, 10.976 g of phosphorus pentachloride are added with stirring to a mixture of 21.477 g (0.092 mol) of 3-cyano-5,7-dihydroxy-6-(sec-butyl)pyrazolo[1,5-a]pyrimidine and 126. 196 g (0.823 mol) of phosphorus oxychloride. The reaction mixture is heated at 110° C. for 3 hours and then concentrated under reduced pressure. The residue that remains is dissolved in dichloromethane. The resulting solution is initially washed with ice-water and then dried over sodium sulfate and concentrated under reduced pressure. The residue that remains is chromatographed on silica gel using petroleum ether:tert-butyl methyl ether=2:1. This gives 9.4 g (33%) of theory) of 3-cyano-5,7-dichloro-6-(sec-butyl)pyrazolo[1,5-a]pyrimidine.

HPLC: logP=3.27

### Example 31

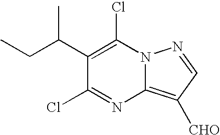

Process (f):

A mixture of 15 g of 5,7-dihydroxy-6-(sec-butyl)pyrazolo[1,5-a]pyrimidine and 35 ml of phosphorus oxychloride is heated under reflux for 1 hour and then cooled to 0° C. 10.6 g of dimethylformamide are then added dropwise with stirring to the reaction mixture such that the temperature of the mixture does not exceed 20° C. After the addition has ended, the mixture is initially stirred at room temperature for 1 hour and then heated under reflux for 2 hours. The mixture is then concentrated under reduced pressure. The residue that remains is stirred with ice-water and the resulting mixture is extracted with ethyl acetate. The combined organic phases are dried over sodium sulfate and then concentrated under reduced pressure. The residue that remains is dissolved in ethyl acetate and filtered through silica gel. This gives 7 g of 3-formyl-5,7-dichloro-6-(sec-butyl)pyrazolo[1,5-a]pyrimidine. The product is used without additional purification for further synthesis.

### Example 32

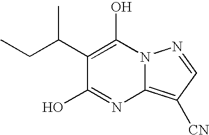

Process (g):

A mixture of 20.0 g (0.092 mol) of diethyl sec-butylmalonate, 9.997 g (0.092 mol) of 4-cyano-5-amino-1H-pyrazole and 18.854 g (0.102 mol) of tri-n-butylamine is heated under reflux at 180° C. for 6 hours. Ethanol liberated during the reaction is continuously distilled off. The reaction mixture is then concentrated under reduced pressure. This gives 21.5 g (100% of theory) of 3-cyano-5,7-dihydroxy-6-(sec-butyl)pyrazolo[1,5-a]pyrimidine. The product is used without additional purification for further syntheses.

The pyrazolopyrimidines of the formula (II) listed in table 2 below are also obtained by the methods described above:

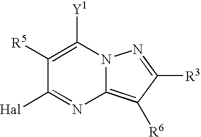

| Ex. | | | | | | |
|---|---|---|---|---|---|---|
| No. | Y¹ | R³ | R⁶ | Hal | R⁵ | logP*) |
| 33 | Cl | H | —COOCH₃ | Cl | 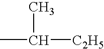 | 3.13 |

### Use Examples

### Example A

Venturia—Test (Apple)/Protective

| | | |
|---|---|---|
| Solvents: | 24.5 | parts by weight of acetone |
| | 24.5 | parts by weight of dimethylacetamide |
| Emulsifier: | 1.0 | part by weight of alkylaryl polyglycol ether |

To produce a suitable preparation of active compound, one part weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple pathin Venturia inaequalis and then remain in an incubation cabinet at about 20° C. and 100% relative atmospheric humidity for one day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the compounds according to the invention listed in examples 1, 3 and 4 showed, at an application rate of 100 g/ha, an efficacy of more than 80%.

### Example B

Botrytis—Test (Bean)/Protective

| | | |
|---|---|---|
| Solvents: | 24.5 | parts by weight of acetone |
| | 24.5 | parts by weight of dimethylacetamide |
| Emulsifier: | 1.0 | part by weight of alkylaryl polyglycol ether |

To produce a suitable preparation of active compound, one part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, two small pieces of agar colonized by Botrytis cinerea are placed onto each leaf. The inoculated plants are placed in a dark chamber at about 20° C. and 100% relative atmospheric humidity.

The size of the infected areas on the leaves is evaluated 2 days after the inoculation. 0% means an efficacy which corresponds to that of the control, whereas an efficacy of 100% means that no infection is observed.

In this test, the compounds according to the invention listed in examples 1 and 4 showed, at an application rate of 500 g/ha, an efficacy of more than 80%.

### Example C

Erysiphe Test (Barley)/Protective

| | | |
|---|---|---|
| Solvent: | 49 | parts by weight of N,N-dimethylacetamide |
| Emulsifier: | 1.0 | part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. 1 day after the treatment, the plants are inoculated with spores of Erysiphe graminis f. sp. hordei. The plants are then placed in a greenhouse at 70% relative atmospheric humidity and a temperature of 18° C.

Evaluation is carried out 7 days after the inoculation. 0% means an efficacy which corresponds to that of the control, whereas an efficacy of 100% means that no infection is observed.

In this test, the compound according to the invention listed in example 3 showed, at an application rate of 750 g/ha, an efficacy of more than 80%.

1. A pyrazolopyrimidine of the formula 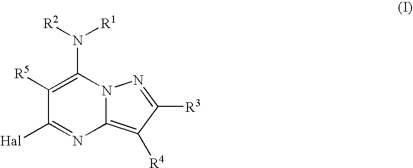
in which
R¹ represents optionally substituted alkyl, optionally substituted alkenyl, optionally substituted alkinyl, optionally substituted cycloalkyl or optionally substituted heterocyclyl,
R² represents hydrogen or alkyl, or
R¹ and R² together with the nitrogen atom to which they are attached represent an optionally substituted heterocyclic ring,
R³ represents hydrogen, halogen, optionally substituted alkyl or optionally substituted cycloalkyl,
R⁴ represents halogen, cyano, nitro, alkyl, hydroxyalkyl, alkoxyalkyl, haloalkyl, cycloalkyl, formyl, thiocarbamoyl, alkoxycarbonyl, alkylcarbonyl, benzylcarbonyl, cycloalkylcarbonyl, hydroximinoalkyl, alkoximinoalkyl, alkylthio, alkylsulfinyl, alkylsulfonyl or alkylaminocarbonyl,
Hal represents halogen and
R⁵ represents alkyl, haloalkyl, alkenyl, haloalkenyl, cycloalkyl, halogen- or methyl-substituted cycloalkyl, cycloalkenyl or halogen- or methyl-substituted cycloalkenyl.

2. The pyrazolopyrimidine of the formula (I) as claimed in claim 1 in which
R¹ represents alkyl having 1 to 6 carbon atoms which may be mono- to pentasubstituted by identical or different substituents selected from the group consisting of halogen, cyano, hydroxy, alkoxy having 1 to 4 carbon atoms and cycloalkyl having 3 to 6 carbon atoms, or
R¹ represents alkenyl having 2 to 6 carbon atoms which may be mono- to trisubstituted by identical or different substituents selected from the group consisting of halogen, cyano, hydroxy, alkoxy having 1 to 4 carbon atoms and cycloalkyl having 3 to 6 carbon atoms, or
R¹ represents alkinyl having 3 to 6 carbon atoms which may be mono- to trisubstituted by identical or different substituents selected from the group consisting of halogen, cyano, alkoxy having 1 to 4 carbon atoms and cycloalkyl having 3 to 6 carbon atoms, or
R¹ represents cycloalkyl having 3 to 6 carbon atoms which may be mono- to trisubstituted by identical or different substituents selected from the group consisting of halogen and/or alkyl having 1 to 4 carbon atoms, or
R¹ represents saturated or unsaturated heterocyclyl having 5 or 6 ring members and 1 to 3 heteroatoms, where the heterocyclyl may be mono- or disubstituted by halogen, alkyl having 1 to 4 carbon atoms, cyano, nitro or cycloalkyl having 3 to 6 carbon atoms,
R² represents hydrogen or alkyl having 1 to 4 carbon atoms, or
R¹ and R² together with the nitrogen atom to which they are attached represent a saturated or unsaturated heterocyclic ring having 3 to 6 ring members, where the heterocycle may contain a further nitrogen, oxygen or sulfur atom as a ring member and where the heterocycle may be substituted up to 3 times by fluorine, chlorine, bromine, alkyl having 1 to 4 carbon atoms or haloalkyl having 1 to 4 carbon atoms and 1 to 9 fluorine and/or chlorine atoms,
R³ represents hydrogen, fluorine, chlorine, bromine, iodine, alkyl having 1 to 4 carbon atoms, haloalkyl having 1 to 4 carbon atoms and 1 to 4 halogen atoms or cycloalkyl having 3 to 6 carbon atoms,
R⁴ represents cyano, fluorine, chlorine, bromine, iodine, nitro, formyl, haloalkyl having 1 to 4 carbon atoms and 1 to 9 fluorine, chlorine or bromine atoms, alkyl having 1 to 4 carbon atoms, hydroxyalkyl having 1 to 4 carbon atoms, alkoxyalkyl having 1 to 4 carbon atoms in the alkoxy moiety and 1 to 4 carbon atoms in the alkyl moiety, cycloalkyl having 3 to 6 carbon atoms, thiocarbomoyl, alkoxycarbonyl having 1 to 4 carbon atoms in the alkoxy moiety, alkylcarbonyl having 1 to 4 carbon atoms in the alkyl moiety, benzylcarbonyl, cycloalkylcarbonyl having 3 to 6 carbon atoms in the cycloalkyl moiety, hydroximinoalkyl having 1 to 4 carbon atoms in the alkyl moiety, alkoximinoalkyl having 1 to 4 carbon atoms in the alkoxy moiety and 1 to 4 carbon atoms in the alkyl moiety, alkylthio having 1 to 4 carbon atoms, alkylsulfinyl having 1 to 4 carbon atoms, alkylsulfonyl having 1 to 4 carbon atoms or alkylaminocarbonyl having 1 to 4 carbon atoms in the alkyl moiety, Hal represents fluorine, chlorine or bromine and
R⁵ represents alkyl having 1 to 6 carbon atoms, alkenyl having 2 to 6 carbon atoms, cycloalkyl having 3 to 8 carbon atoms, cycloalkenyl having 3 to 8 carbon atoms, haloalkyl having 1 to 6 carbon atoms and 1 to 5 fluorine, chlorine or bromine atoms, haloalkenyl having 2 to 6 carbon atoms and 1 to 5 fluorine, chlorine or bromine atoms, cycloalkyl which has 3 to 8 carbon atoms and is substituted by 1 to 3 fluorine, chlorine or bromine atoms or cycloalkenyl which has 3 to 8 carbon atoms and is substituted by 1 to 3 fluorine, chlorine or bromine atoms.

3. The pyrazolopyrimidine of the formula (I) as claimed in claim 1 in which
R¹ represents a radical of the formula 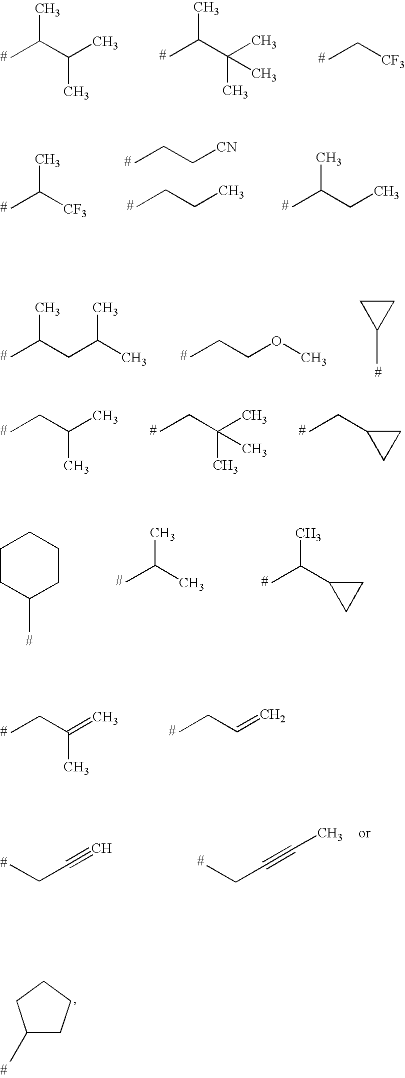
where # denotes the point of attachment,
R² represents hydrogen, methyl, ethyl or propyl, or
R¹ and R² together with the nitrogen atom to which they are attached represent pyrrolidinyl, piperidinyl, morpholinyl, thiomorpholinyl, piperazinyl, 3,6-dihydro-1(2H)-piperidinyl or tetrahydro-1(2H)-pyridazinyl, where these radicals may be substituted by 1 to 3 fluorine atoms, 1 to 3 methyl groups or trifluoromethyl, or
R¹ and R² together with the nitrogen atom to which they are attached represent a radical of the formula 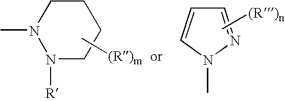
in which
R′ represents hydrogen or methyl,
R″ represents methyl, ethyl, fluorine, chlorine or trifluoromethyl,
m represents the numbers 0, 1, 2 or 3, where R″ represents identical or different radicals, if m represents 2 or 3,
R′″ represents methyl, ethyl, fluorine, chlorine or trifluoromethyl and
n represents the numbers 0, 1, 2 or 3, where R′″ represents identical or different radicals if n represents 2 or 3,
R³ represents hydrogen, fluorine, chlorine, bromine, iodine, methyl, ethyl, isopropyl, cyclopropyl, cyclobutyl, cyclopentyl, cyclohexyl, trifluoromethyl, 1-trifluoromethyl-2,2,2-trifluoroethyl or hepta-fluoroisopropyl,
R⁴ represents cyano, fluorine, chlorine, bromine, iodine, nitro, formyl, trifluoromethyl, difluoromethyl, chloromethyl, methyl, ethyl, cyclopropyl, thiocarbamoyl, methoxycarbonyl, methylcarbonyl, ethylcarbonyl, benzylcarbonyl, cyclopropylcarbonyl, cyclopentylcarbonyl, cyclohexylcarbonyl, hydroximinomethyl, methoximinomethyl, methylthio, methylsulfinyl, methylsulfonyl, methylaminocarbonyl, hydroxymethyl, hydroxyeth-1-yl, methoxymethyl, ethoxymethyl or 1-methoxyethyl,
Hal represents fluorine or chlorine and
R⁵ represents alkyl having 1 to 4 carbon atoms, alkenyl having 2 to 4 carbon atoms, cycloalkyl having 3 to 7 carbon atoms or cycloalkenyl having 3 to 7 carbon atoms, or
R⁵ represents haloalkyl having 1 to 4 carbon atoms and 1 to 5 fluorine, chlorine or bromine atoms, haloalkenyl having 3 or 4 carbon atoms and 1 to 3 fluorine, chlorine or bromine atoms, cycloalkyl which has 3 to 6 carbon atoms and substituted by 1 to 3 fluorine, chlorine or bromine atoms or cycloalkenyl which has 3 to 6 carbon atoms and is substituted by 1 to 3 fluorine, chlorine or bromine atoms.

4. The pyrazolopyrimidine of the formula (I) as claimed in claim 1, in which
R³ represents hydrogen, fluorine, chlorine, bromine, methyl, ethyl, propyl, isopropyl, trifluoromethyl or cyclopropyl and
R⁵ represents methyl, ethyl, propyl, isopropyl, n-butyl, i-butyl, sec-butyl, tert-butyl, allyl, but-2-en-1-yl, cyclopropyl, cyclopentyl, cyclohexyl, cyclopentenyl, cyclohexenyl, chloromethyl, trifluoromethyl, trifluoroisopropyl, trichloroallyl, 2,2-dichlorocyclopropyl or dichloro-cyclohexenyl.

5. A process for preparing pyrazolopyrimidines of the formula (I) as claimed in claim 1, comprising characteried in that
(a) reacting halopyrazolopyrimidines of the formula 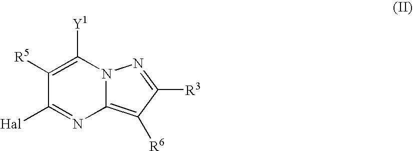
in which
R³, R⁵ and Hal are as defined in claim 1,
R⁶ represents halogen, cyano, nitro, alkyl, haloalkyl, cycloalkyl, formyl, thiocarbamoyl, alkoxycarbonyl, alkylthio, alkylsulfinyl, alkylsulfonyl or alkylaminocarbonyl and
Y¹ represents halogen,
with amines of the formula 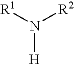
in which
R¹ and R² are as defined in claim 1,
optionally in the presence of a diluent, optionally in the presence of an acidic receptor and optionally in the presence of a catalyst, or
b) reacting pyrazolopyrimidines of the formula 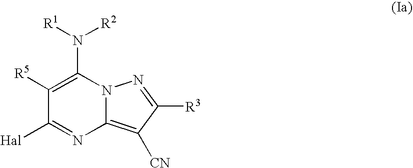
in which
R¹, R², R³, R⁵ and Hal are as defined in claim 1 with either
α) diisobutylaluminum hydride in the presence of aqueous ammonium chloride solution and in the presence of an organic diluent, or
β) Grignard compounds of the formula
R⁷—Mg—X² (IV)
in which
R⁷ represents alkyl, benzyl or cycloalkyl and
X² represents chlorine, bromine or iodine,
in the presence of a diluent and, optionally, in the presence of a catalyst, or
c) reacting pyrazolopyrimidines of the formula 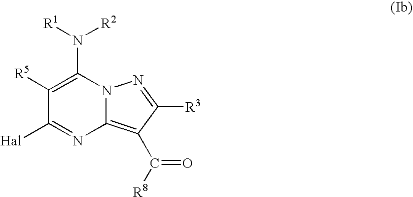
in which
(a) R¹, R², R³, R⁵ and Hal are as defined above and
R⁸ represents hydrogen, alkyl, benzyl or cycloalkyl, with either
α) amino compounds of the formula
H₂N—OR⁹ (V)
in which
R⁹ represents hydrogen or alkyl,
in the presence of a diluent and, optionally, in the presence of a catalyst, where the amino compounds of the formula (V) can also be employed in the form of their acid addition salts, or
β) diisobutylaluminum hydride in the presence of aqueous ammonium chloride solution and in the presence of an organic diluent, or sodium borohydride in the presence of a diluent, and reacting the resulting pyrazolopyrimidines of the formula 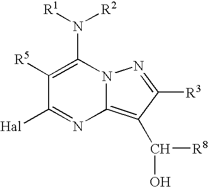
in which
R¹, R², R³, R⁵, R⁸ and Hal are as defined above with alkylating agents of the formula
R¹⁰—X¹
in which
R¹⁰ represents alkyl and
X¹ represents chlorine, bromine, iodine or the radical R⁸O—SO₂—O—, optionally in the presence of a base and in the presence of a diluent,
d) reacting pyrazolopyrimidines of the formula 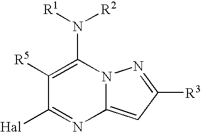
in which
R¹, R², R³, R⁵ and Hal are as defined above with acid halides of the formula 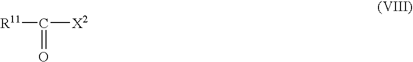
in which
R¹¹ represents alkyl, benzyl or cycloalkyl and
X² represents chlorine or bromine, or
with acid anhydrides of the formula 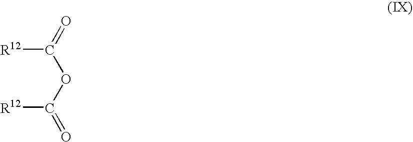
in which
R¹² represents alkyl, in each case in the presence of a catalyst and in the presence of a diluent.

6. A composition for controlling unwanted microorganisms, comprising at least one pyrazolopyrimidine of the formula (I) according to claim 1, and extenders or surfactants.

7. The composition as claimed in claim 6, comprising at least one further fungicidally or insecticidally active compound.

8. (canceled)

9. A method for controlling unwanted microorganisms, comprising contacting unwanted microorganisms or their habitat with pyrazolopyrimidines of the formula (I) as claimed in claim 1.

10. A process for preparing compositions for controlling unwanted microorganisms, comprising mixing a pyrazolopyrimidine of the formula (I) as claimed in claim 1 with extenders or surfactants.
